(19) United States
(12) Patent Application Publication
(10) Pub. No.: US 2011/0160640 A1
(43) Pub. Date: Jun. 30, 2011

(54) Operation management of active transdermal medicament patch

(76) Inventor: Jamal S. Yanaki, Salt Lake City, UT (US)

(21) Appl. No.: 12/927,691

(22) Filed: Nov. 19, 2010

Related U.S. Application Data
(63) Continuation-in-part of application No. 12/009,443, filed on Jan. 18, 2008.

(51) Int. Cl. A61N 1/30

(52) U.S. Cl. 604/20

(57) ABSTRACT

A transdermal medicament patch includes a flexible substrate having a therapeutic face configured for releasable retention against the skin of a patient, a medicament matrix susceptible to permeation by medicament and secured to the therapeutic face of the substrate, and a return electrode secured to the therapeutic face spaced from the medicament matrix. The return electrode and the medicament matrix effect electrically conductive engagement with the skin of the patient when the substrate is retained thereupon. A power source is carried on substrate so electrically coupled between the medicament matrix and the return electrode as to cause iontophoretic migration of medicament from the medicament matrix into the skin of the patient. A dosage control circuit carried non-removably on said substrate limits to a predetermined medicament quantity the total medicament administered into the skin of the patient by iontophoretic migration during a predetermined therapy period.

### RELATED APPLICATIONS

This is a continuation-in-part patent application of pending U.S. patent application Ser. No. 12/009,443 that was filed on Jan. 18, 2008.

### BACKGROUND

1. Field of the Invention

The invention disclosed herein relates to the transdermal administration of medicaments to human and animal subjects. More particularly, the present invention pertains to active iontophoretic delivery systems in which electrical contacts are applied to the surface of the skin of a subject for the purpose of delivering medicament through the surface of the skin into underlying tissue.

2. Background Art

During active iontophoresis, direct electrical current is used to cause ions of a soluble medicament to move across the surface of the skin and to diffuse into underlying tissue. The surface of the skin is not broken by this administration of the medicament. When conducted within appropriate parameters, the sensations experienced by a subject during the delivery of the medicament in this manner are not unpleasant. Therefore, active iontophoresis presents an attractive alternative to hypodermic injections and to intravascular catheterization.

The direct current employed in active iontophoresis systems may be obtained from a variety of electrical power sources. These include consumable and rechargeable batteries, paired regions of contrasting galvanic materials that when coupled by a fluid medium produce minute electrical currents, and electrical equipment that ultimately receives power from a wall socket. The later in particular are of such bulk, weight, and cost as to necessitate being configured as items of equipment distinct from the electrical contacts that are applied directly to the skin in administering a medicament iontophoretically. Accordingly, such power sources limit the mobility of the patient during the time that treatment is in progress.

A flow of electrical current requires an uninterrupted, electrically-conductive pathway from the positive pole of a power source to the other, negative pole thereof. Living tissue is made up primarily of fluid and is, therefore, a conductor of electrical current. In an iontophoretic circuit, the opposite poles of a power source are electrically coupled to respective, separated contact locations on the skin of the subject. The difference in electrical potential created by the power source between those contact locations causes a movement of electrons and electrically charged molecules, or ions, through the tissue between the contact locations.

In an active iontophoretic delivery system, the polarity of the net overall electrical charge on dissolved molecules of a medicament determines the nature of the electrical interconnection that must be effected between the power source that is used to drive the system and the supply of medicament that is positioned on the skin of the patient at one of the contact locations to be used by the system. A positively charged medicament in a reservoir against the skin of a patient is coupled to the positive pole of the power source that is to be used to administer the medicament iontophoretically. Correspondingly, a reservoir on the skin of a patient containing a negatively charged medicament must be coupled to the negative pole of such a power source. Examples of common iontophoretically administrable medicaments in each category of polarity are listed in the table below.

| Positive Polarity Medicaments | Negative Polarity Medicaments |
|---|---|
| Bupivacaine hydrochloride | Acetic acid |
| Calcium chloride | Betamethasone sodium phosphate |
| Lidocaine hydrochloride | Copper sulfate |
| Zinc chloride | Dexamethasone sodium phosphate |
| Lidocaine | Fentinol |
| | Magnesium sulfate |
| | Naproxen sodium |
| | Sodium chloride |
| | Sodium salicylate |
| | Ascorbic acid |
| | Hydroquinone |
| | Vitamins A, C, D, or E |

The medicament is housed in a fluid reservoir, or medicament, which is then positioned electrically conductively engaging the skin of the subject at an anatomical location overlying the tissue to which the medicament is to be administered. The medicament matrix can take the form of a gel suspension of the medicament or of a pad of an absorbent material, such as gauze or cotton, which is saturated with fluid containing the medicament. In some instances the fluid containing the medicament is provided from the manufacturer in the absorbent pad. More commonly, the fluid is added to the absorbent pad by a medical practitioner at the time that the medicament is about to be administered to a subject.

An iontophoretic circuit for driving the medicament through the unbroken skin is established by coupling the appropriate pole of the power source through the medicament matrix to the skin of the subject at the anatomical location at which the medicament is to be administered. Simultaneously, the other pole of the power source is coupled to an anatomical location on the skin of the subject that is distanced from the medicament matrix. The coupling of each pole of the power source is effected by the electrical connection of each pole to a respective electrode. The electrode at the medicament matrix is referred to as an active electrode; the electrode at the contact location on the skin distanced from the medicament matrix is referred to as a return electrode.

The medicament matrix with an associated active electrode may be conveniently retained against the skin by a first adhesive patch, while the return electrode may be retained against the skin at some distance from the medicament matrix using a distinct second adhesive patch. Alternatively, the medicament matrix with the associated active electrode, as well as the return electrode, may be carried on a single adhesive patch at, respective, electrically isolated locations.

The use of iontophoresis to administer medicaments to a subject is advantageous in several respects.

Medications delivered by an active iontophoretic system bypass the digestive system. This reduces digestive tract irritation. In many cases, medicaments administered orally are less potent than if administered transcutaneously. In compensation, it is often necessary in achieving a target effective dosage level to administer orally larger quantities of medicament than would be administered transcutaneously.

Active iontophoretic systems do not require intensive skin site sanitation to avoid infections. Patches and the other equipment used in active iontophoresis do not interact with bodily fluids and, accordingly, need not be disposed as hazardous biological materials following use. Being a noninvasive procedure, the administration of medicament using an active iontophoretic system does not cause tissue injury of the types observed with hypodermic injections and with intravenous catheterizations. Repeated needle punctures in a single anatomical region, or long term catheter residence, can adversely affect the health of surrounding tissue. Needle punctures and catheter implantations inherently involve the experience of some degree of pain. These unintended consequences of invasive transcutaneous medicament administration are particularly undesirable in an area of the body that, being already injured, is to be treated directly for that injury with a medicament. Such might be the case, for example, in the treatment of a strained muscle or tendon.

With some exceptions, no pharmacologically significant portion of a medicament delivered iontophoretically becomes systemically distributed. Rather, a medicament delivered iontophoretically remains localized in the tissue at the site of administration. This minimizes unwanted systemic side effects, reduces required dosages, and lightens the burdens imposed on the liver and kidneys in metabolizing the medicament.

The dosage of a medicament delivered iontophoretically is conveniently and accurately measured by monitoring the amount and the duration of the current flowing during the administration. With current being measured in amperes and time being measured in minutes, the dosage of medicament given transcutaneously is given in units of ampere-minutes. Due to the minute quantities of medicament required in active iontophoresis, medicament dosage in active iontophoresis is generally prescribed in milliamp-minutes. Dosage measured in this manner is more precise than is dosage measured as a fluid volume or as a numbers of tablets.

Finally, the successful operation of an active iontophoretic system is not reliant in any significant respect on the medical skills of nurses or doctors. Foregoing the involvement of such medical personnel in the administration of medicaments, whenever appropriate, favors the convenience of patients and reduces the costs associated with the delivery of such types of therapy.

### SUMMARY OF THE INVENTION

The present invention promotes the wide use of active iontophoretic systems by providing improved components and combinations of components for active iontophoretic systems. The present invention thus improves the safety of patients and reduces the technical difficulty of related tasks that must by performed by medical personnel.

The teachings of the present invention enhance the reliability and the user friendliness of active iontophoretic systems and lead to reductions in the costs associated with the manufacture of such systems, as well as with the use of such systems to deliver medication.

While selected aspects of the present invention have applicability in all types of active iontophoretic systems, including those that employ plural disposable adhesive patches in combination with reusable power sources and controls, the teachings of the present invention are most optimally applicable to such system as involve a single fully-integrated, active transdermal medicament patch.

Thus, in one aspect of the present invention, a fully-integrated, independently accurately performing adhesive active transdermal medicament patch is provided.

The present invention contemplates related methods of design and manufacture, as well as methods pertaining to the treatment of patient health problems.

Additional objects and advantages of the invention will be set forth in the description which follows, and in part will be obvious from the description, or may be learned by the practice of the invention. The objects and advantages of the invention may be realized and obtained by means of the instruments and combinations particularly pointed out in the appended claims.

### BRIEF DESCRIPTION OF THE DRAWINGS

The manner in which the above-recited and other advantages and objects of the invention are obtained will be understood by a more particular description of the invention rendered by reference to specific embodiments thereof that are illustrated in the appended drawings. These figures are intended to be illustrative, not limiting. Although the invention is generally described in the context of these embodiments, it should be understood that by so doing, no intention exists to limit the scope of the invention to those particular embodiments.

Understanding that these drawings depict only typical embodiments of the invention and are not therefore to be considered limiting of scope, the invention will be described and explained with additional specificity and detail through the use of the accompanying drawings in which:

FIG. 1 is a perspective view of an embodiment of a fully-integrated, active transdermal medicament patch incorporating teachings of the present invention being worn during activity by a patient requiring the localized administration of a medicament;

FIG. 2A is a perspective view of the active transdermal medicament patch of FIG. 1 showing the substrate of the patch, a moistened medicament matrix mounted on the therapeutic face of the substrate that engages the skin of the patient in FIG. 1, and a release liner in the process of being peeled from an adhesive coating on the portion of the therapeutic face not occupied by the medicament matrix;

FIG. 2B is a perspective view of the active transdermal medicament patch of FIG. 2A with the release liner illustrated in FIG. 2A fully removed;

FIG. 2C is a partially-exploded perspective view of the active transdermal medicament patch of FIG. 2B that reveals the entirety of the therapeutic face of the substrate of the medicament patch;

FIG. 3A is a perspective view of the active transdermal medicament patch of FIG. 1 taken from the side thereof visible in FIG. 1, the side opposite that illustrated in FIGS. 2A-2C;

FIG. 3B is an exploded perspective view of the active transdermal medicament patch of FIG. 3A showing the cover of the medicament patch, the upper face of the substrate of the medicament patch, and a circuit board sandwiched therebetween in a folded, compact state;

FIG. 3C is a perspective view of the circuit board of FIG. 3B in a partially-unfolded state thereof;

FIG. 3D is a partially-exploded perspective view of the circuit board of FIG. 3C in a fully-unfolded, planar state thereof;

FIG. 4 is a cross-sectional elevation view of the active transdermal medicament patch of FIG. 2A taken along section line 4-4 shown therein;

FIG. 5A is cross-sectional elevation view of the active transdermal medicament patch of FIG. 4 inverted and disposed against the skin of a patient, thereby to illustrate the movement of a medicament of positive polarity through subcutaneous tissue of the patient;

FIG. 5B is a diagram like that of FIG. 5A, illustrating the movement of a medicament of negative polarity through subcutaneous tissue of a patient;

FIG. 6 is a diagram like FIG. 5B reversed in left-right orientation and illustrating the movement of a medicament of negative polarity through subcutaneous tissue of a patient caused by the active transdermal medicament patch of FIG. 1;

FIG. 7 is a simplified rendering of FIG. 6 depicting primarily functional elements of the circuit shown therein;

FIG. 8 is a schematic diagram of an embodiment of electronics incorporating teachings of the present invention and suitable for use in the active transdermal medicament patch of FIG. 7;

FIGS. 9A and 9B are the same performance curve, but drawn in contrasting respective scales, of a first performance parameter of the electronics of FIG. 8 taken over a predetermined therapy period;

FIGS. 10A and 10B are the same performance curve, but drawn in contrasting respective scales, of a second performance parameter of the electronics of FIG. 8 taken over the same predetermined therapy period used in FIGS. 9A and 9B;

FIG. 11 is a performance curve of a third performance parameter of the electronics of FIG. 8 taken over the same predetermined therapy period used in FIGS. 9A-9B and 10A-10B;

FIG. 12 is a flowchart illustrating selected steps performed by the electronics of FIG. 8;

FIG. 13A is an anticipated performance curve of the voltage applied to the skin of a patient by the electronics of FIG. 8 throughout a single periodic voltage-sampling cycle at the initiation of operation, the voltage-sampling cycle being conducted to determine the electrical current flow resistance through the skin between the medicament matrix and the return electrode of the active transdermal medicament patch of FIG. 1;

FIG. 13B is an actual performance curve of the voltage applied to the skin of a patient under the conditions described relative to FIG. 13A;

FIG. 14 is a diagram depicting the capacitance understood to arise between the skin of a patient and the electrical contacts of the active transdermal medicament patch of FIG. 1, a phenomenon that accounts for the delay in actual voltage sampling depicted in FIG. 13B; and

FIG. 15 is an illustrative performance curve of the voltage applied to the skin of a patient over a succession of typical voltage-sampling cycles of the type shown in FIG. 13B during the progression of electroporation at the initiation of the operation of the active transdermal medicament patch of FIG. 1.

### DETAILED DESCRIPTION OF THE INVENTION

In the following description, for purpose of explanation, specific details are set forth in order to provide an understanding of the invention. Nonetheless, the present invention may be practiced without some or all of these details. The embodiments of the present invention, some of which are described below, may be incorporated into a number of elements of medical systems additional to the medical systems in which those embodiments are by way of necessity illustrated herein. Structures and devices shown in the figures illustrate merely exemplary embodiments of the present invention, thereby to facilitate discussion of teachings of the present invention. Thus, the details of the structures and devices shown in the figures are not supplied herein in order to serve detractors as instruments with which to mount colorable denials of the existence of broad teachings of present invention that are manifest from this specification taken as a whole.

Connections between components illustrated in the figures are not limited to direct connections between those components. Rather, connections between such components may be modified, reformatted, or otherwise changed to include intermediary components without departing from the teachings of the present invention.

References in the specification to “one embodiment” or to “an embodiment” mean that a particular feature, structure, characteristic, or function described in connection with the embodiment being discussed is included in at least one embodiment of the present invention. Furthermore, the use of the phrase “in one embodiment” in various places throughout the specification is not necessarily a reference in each instance of use to any single embodiment of the present invention.

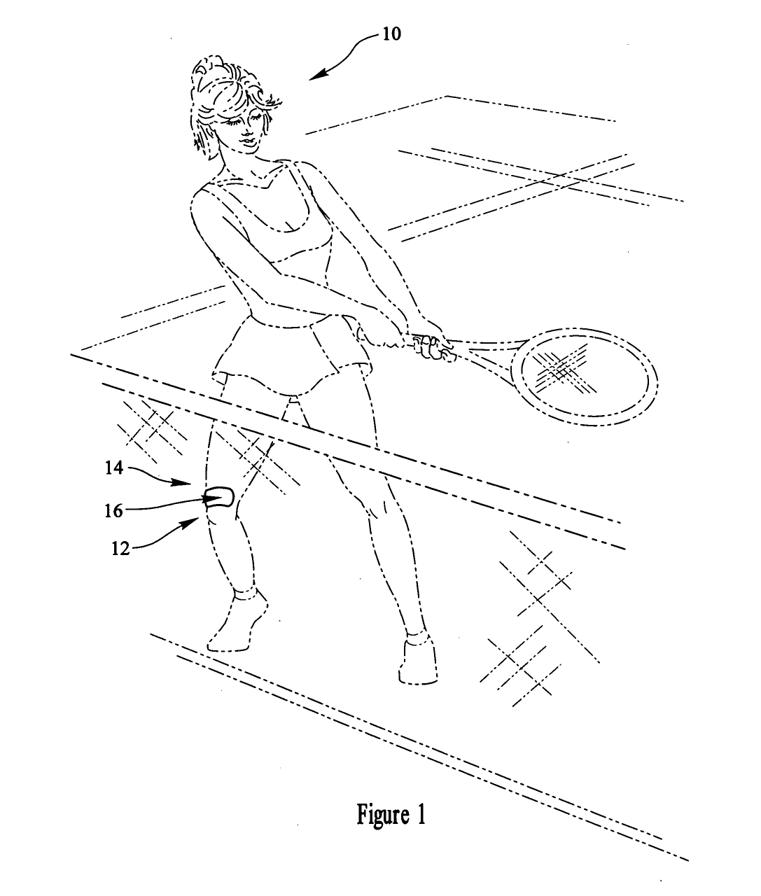

FIG. 1 shows a patient 10 requiring the localized administration of a medicament to knee 12 thereof. For that purpose, patient 10 is wearing on knee 12 thereof one embodiment of an active iontophoretic delivery system 14 that incorporates teachings of the present invention. While so doing, patient 10 is nonetheless able to engage in vigorous physical activity, because delivery system 14 is entirely self-contained, and not supplied with power from any immobile or cumbersome power source. Delivery system 14 takes the form of a fully-integrated, active transdermal medicament patch 16 that is removable adhered to the skin of knee 12 of patient 10 for the duration of a predetermined therapy period. The length of the therapy period during which medicament patch 16 must be worn is determined by the rate at which medicament patch 16 delivers medicament through the skin of patient 10 and the total dose of medicament that is to be administered.

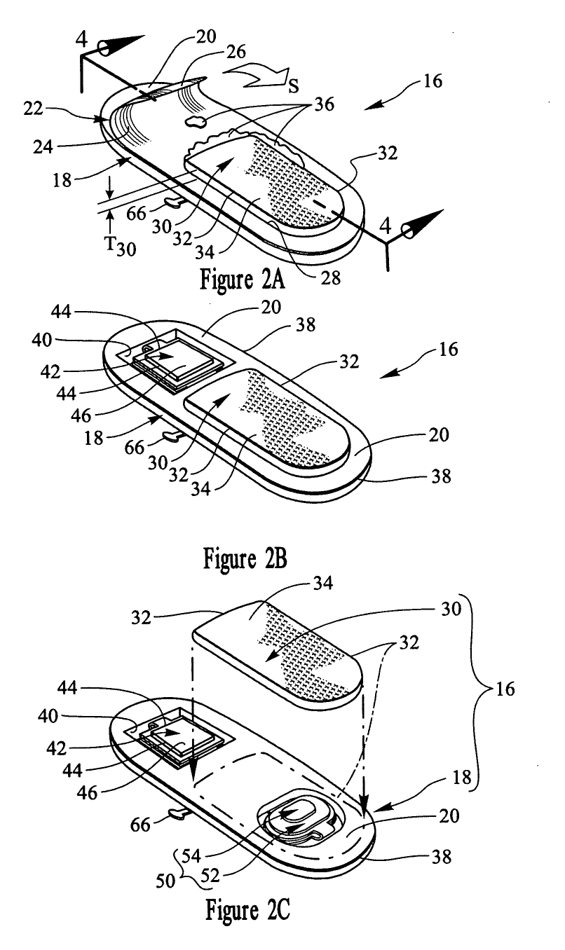

FIGS. 2A-4 taken together afford an understanding of the relationships existing among the structural elements of medicament patch 16.

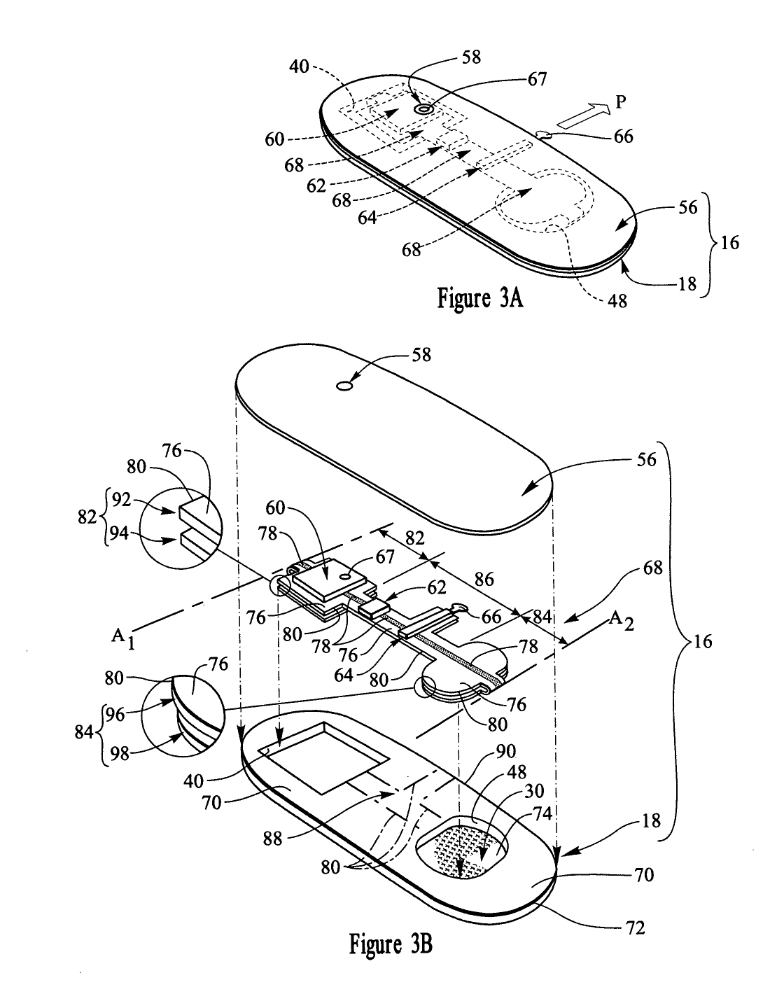

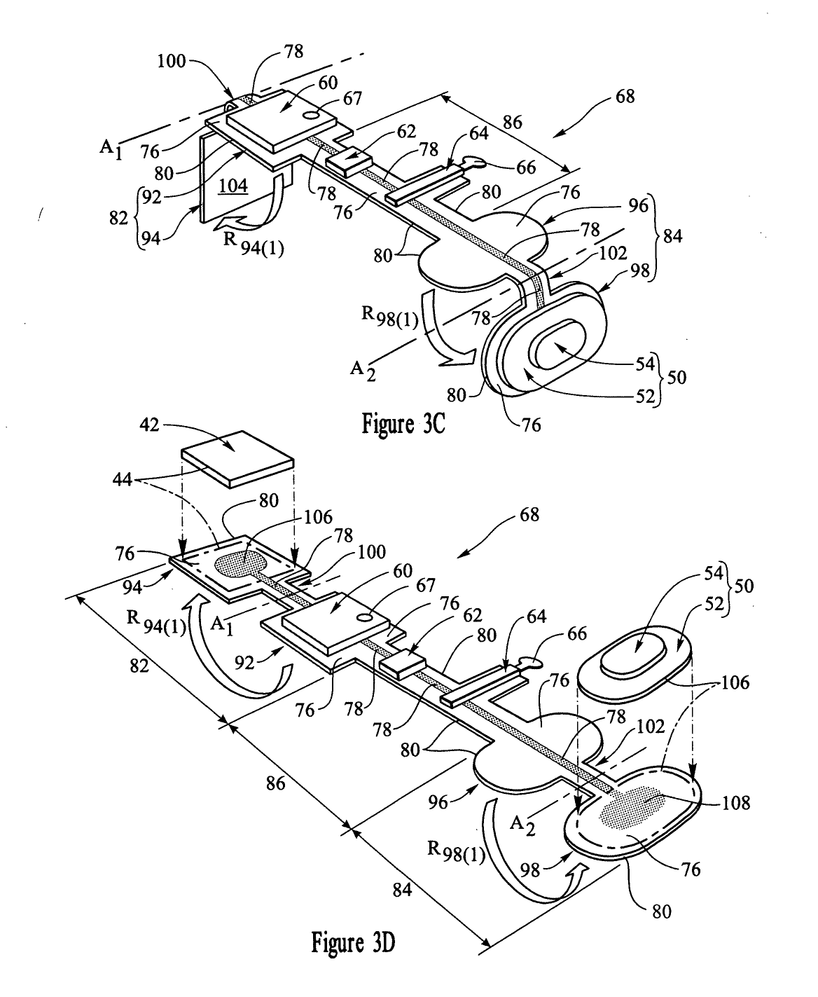

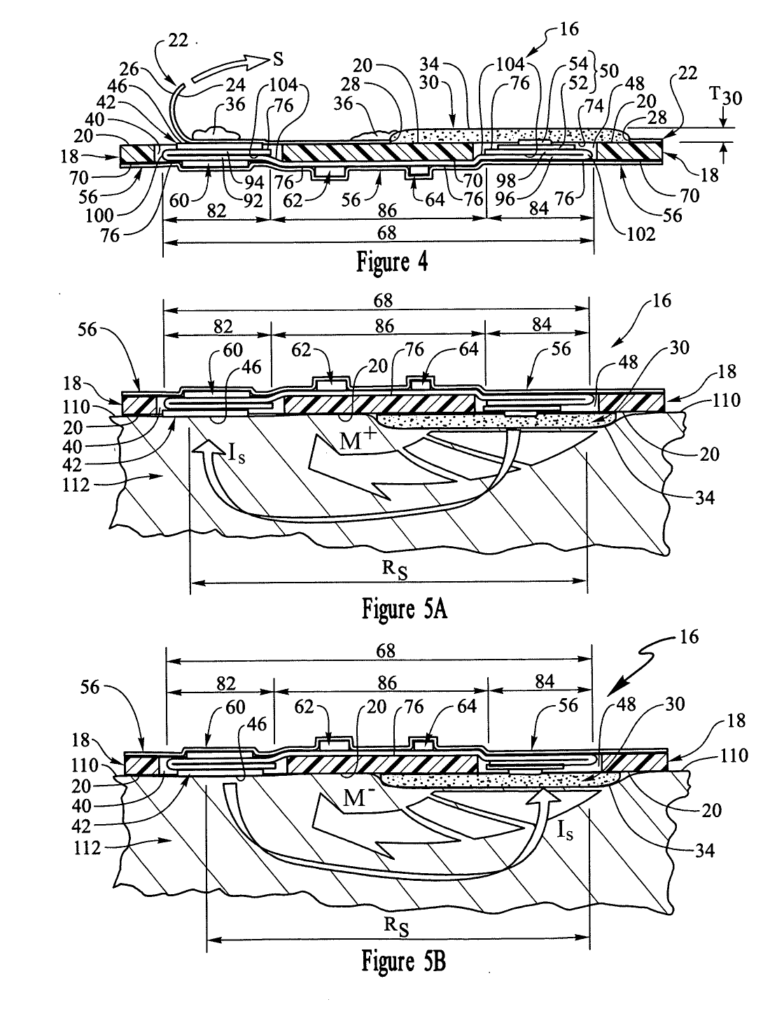

FIGS. 2A-2C are views in various stages of disassembly of the side of medicament patch 16 that engages the skin of patient 10 in FIG. 1. FIGS. 3A-3D are similar views of the opposite side of medicament patch 16, the side thereof visible in FIG. 1. FIG. 4 is a cross-sectional elevation view of medicament patch 16 taken along section line 4-4 in FIG. 2A.

FIG. 2A reveals that medicament patch 16 includes a flexible, planar electrically non-conductive biocompatible substrate 18 having a therapeutic face 20 on one side thereof that is intended to be disposed in contact with the skin of a patient, such as patient 10 in FIG. 1. Therapeutic face 20 is coated with a biocompatible adhesive to a sufficient extent as will enable therapeutic face 20 to be removably secured to the skin of patient 10. Prior to the actual use of medicament patch 16, the adhesive on therapeutic face 20 is shielded by a removable release liner 22. As suggested by arrow S in FIG. 2A, release liner 22 is in the process of being peeled from therapeutic face 20. Release liner 22 has on the opposite sides thereof, respectively, first an exposed face 24 and second a contact face 26 that actually engages the adhesive on therapeutic face 20 of substrate 18.

Formed generally centrally through release liner 22 is a medicament matrix aperture 28. As shown in FIG. 2A, medicament matrix aperture 28 is substantially filled by a generally planar medicament matrix 30 that exhibits a periphery 32 that closely conforms in shape and size to the shape and size of medicament matrix aperture 28. Medicament matrix 30 can take the form of a gel suspension permeated by medicament, but as illustrated in FIG. 2A, medicament matrix 30 is an absorbent pad of gauze or cotton that is saturated by a user with a fluid solution containing the medicament just prior to the use of medicament patch 16. In some instances, medicament patch 16 is supplied by the manufacturer with medicament solution already permeating medicament matrix 30.

The side of medicament matrix 30 visible in FIG. 2A has a periphery 32 that encloses a skin contact surface 34 of medicament matrix 30. Medicament matrix 30 projects through medicament matrix aperture 28 in such a manner that skin contact surface 34, while oriented generally parallel to the plane of release liner 22 and the plane of therapeutic face 20 of substrate 18, is separated from each by a distance that is approximately equal to the thickness T₃₀ of medicament matrix 30. Skin contact surface 34 of medicament matrix 30 electrically conductively engage the skin of patient 10, when therapeutic face 20 of substrate 18 is disposed against and removably adhered thereto.

By way of example, the embodiment of medicament matrix 30 shown in FIG. 2A is an absorbent pad that must become permeated by a medicament solution before use. The saturation of medicament matrix 30 with medicament solution 36 is a process intended to be performed by medical personnel just prior to the disposition of medicament patch 16 against the skin of a patient.

FIG. 2A reveals that in such a process, drops of a medicament solution 36 may inadvertently be deposited on exposed face 24 of release liner 22 remote from medicament matrix 30. Also, at various locations about periphery 32 of medicament matrix 30, further drops of medicament solution 36 may be expected to overflow onto exposed face 24 of release liner 22 due to an over-saturation of portions of medicament matrix 30 with medicament solution 36. Such drops of medicament solution 36 do not, however, contact the adhesive on therapeutic face 20 of substrate 18. Instead, the drops of medicament solution 36 rest upon release liner 22 and are removed from medicament patch 16 with release liner 22, when release liner 22 is pealed from therapeutic face 20 of substrate 18 in the manner suggested by arrow S.

FIG. 2B shows therapeutic face 20 of medicament patch 16 after the complete removal of release liner 22 therefrom. There it can bee seen that therapeutic face 20 of medicament patch 16 has a periphery 38 and that medicament matrix 30 is positioned on therapeutic face 20 at one end of substrate 18 interior of periphery 38. Formed through the opposite end of substrate 18 at a position separated from medicament matrix 30 is a first electrode aperture 40. The size and shape of each of substrate 18, medicament matrix 30, and first electrode aperture 40 can vary from those depicted without departing from teachings of the present invention.

Accessible from therapeutic face 20 through first electrode aperture 40 is a planar first electrode, a return electrode 42 of medicament patch 16. Return electrode 42 has a periphery 44 and, interior thereof on the side of return electrode 42 visible in FIG. 2B, a skin contact surface 46. While possible to do so, return electrode 42 is not secured directly to therapeutic face 20 of substrate 18 in the manner of medicament matrix 30. Instead, return electrode 42 is maintained in a fixed relationship to other features of medicament patch 16 with the plane of skin contact surface 46 of return electrode 42 parallel to and closely coincident with the plane of therapeutic face 20. Consequently, a first electrode, such as return electrode 42, will routinely be characterized herein as being carried or positioned on therapeutic face 20, and thereby being located on the same side of substrate 18 as medicament matrix 30.

Return electrode 42 is separated from medicament matrix 30, and thus electrically isolated therefrom. Skin contact surface 46 of return electrode 42 electrically conductively engages the skin of patient 10, when therapeutic face 20 of substrate 16 is disposed against and removable adhered thereto. Accordingly, when medicament patch 16 is adhered to the skin of patient 10 as shown in FIG. 1, return electrode 42 engages the skin of patient 10 at a location that is remote from the location engaged by medicament matrix 30.

FIG. 2C is a partially-exploded perspective view of medicament patch 16 of FIG. 2B. Medicament matrix 30 is depicted above and separated from therapeutic face 20 of substrate 18. Revealed thereby is a second electrode aperture 48 that is formed through substrate 18 at a position separated from first electrode aperture 40 and, correspondingly, also from return electrode 42. Superimposed by way of reference in phantom on therapeutic face 20 is periphery 32 of medicament matrix 30, which in the assembled condition of medicament patch 16 shown in FIG. 2B entirely obscures second electrode aperture 48.

Accessible from therapeutic face 20 through electrode aperture 44 is a planar second electrode, active electrode 50 of medicament patch 16. Active electrode 50 includes an electrically-conductive planar backing layer 52 and a smaller electrically-conductive planar pH-control layer 54 disposed centrally thereupon. While possible to do so, active electrode 50 is not secured directly to therapeutic face 20 of substrate 18 in the manner of medicament matrix 30. Instead, by the attachment of active electrode 50 to other structural elements of medicament patch 16, active electrode 50 is maintained in a fixed relationship to other features of medicament patch 16 with the plane of each of backing layer 52 and pH-control layer 54 parallel to and closely coincident with the plane of therapeutic face 20. Consequently, a second electrode, such as active electrode 50, will routinely be characterized herein as being carried or positioned on therapeutic face 20, and thereby being located on the same side of substrate 18 as, for example, return electrode 42 and medicament matrix 30.

In the assembled condition of medicament patch 16 shown in FIG. 2B, the side of medicament matrix 30 opposite from skin contact surface 34, which is therefore not visible in FIG. 2B, rests against and may be secured to each of backing layer 52 and pH-control layer 54 of active electrode 50. This is borne out in FIG. 2C, where pH-control layer 54 is shown carried on backing layer 52, while each of these components of active electrode 50 are located interior of periphery 32 of medicament matrix 30 as superimposed in phantom on therapeutic face 20.

FIG. 3A is a perspective view of medicament patch 16 taken from the side thereof visible in FIG. 1 when being worn by patient 10, the side of medicament patch 16 opposite that illustrated in FIGS. 2A-2C. The side of medicament patch 16 shown in FIG. 3A is encased in a protective cover 56 that is, but need not be, coextensive with substrate 18 of medicament patch 16. By way of example, cover 56 is depicted as being opaque and as including as the sole transparent portion thereof a small observation port 58. Consequently, features of medicament patch 16 beneath cover 56, such as first electrode aperture 40 and second electrode aperture 48, are shown in dashed lines.

Also included in dashed lines in FIG. 3A are some components of medicament patch 16 that are carried on substrate 18 beneath cover 56. These include electronic circuitry 60, a power source 62, and a user switch 64. User switch 64 is depicted by way of example as a user-operated pull tab switch that permits the initiation of the operation of power source 62 by withdrawing an activation stem 66 of user switch 64 from between cover 56 and substrate 18 in a manner suggested by arrow P. Electronic circuitry 60 is surmounted by a light-emitting diode 67 or other visual indicator that communicates to a user information about the operative status of medicament patch 16. Light-emitting diode 67 is therefore located beneath and in alignment with observation port 58 in cover 56.

Electronic circuitry 60, power source 62, and user switch 64 are not mounted directly to substrate 18, although any or all of these components of medicament patch 16 may be secured directly to substrate 18, or recessed in whole or in part into substrate 18. Instead, electronic circuitry 60, power source 62, and user switch 64 are maintained in a fixed relationship to each other by being commonly secured to a circuit board 68. Circuit board 68 directly engages substrate 18 beneath cover 56, indirectly fixing each of electronic circuitry 60, power source 62, and user switch 64 relative to each other and to other features of medicament patch 16.

Circuit board 68 will be explored in greater detail in FIGS. 3B-3D.

FIG. 3B is an exploded perspective view of medicament patch 16 of FIG. 3A. Cover 56 is depicted above and separated from substrate 18. Revealed thereby is an upper face 70 of substrate 18. Upper face 70 has a periphery 72 that is substantially similar in size and shape to periphery 38 of therapeutic face 20 of substrate 18 shown in FIGS. 2B and 2C on the opposite side of substrate 18 from upper face 70. First electrode aperture 40 and second electrode aperture 48 are formed through substrate 18 at spaced-apart locations. Visible through second electrode aperture 48 is medicament matrix 30 and a portion of a securement surface 74 thereof. Medicament matrix 30 closes the side of second electrode aperture 48 that opens onto therapeutic face 20 of substrate 18. This is the situation when securement surface 74 of medicament matrix 30 engages therapeutic face 20 as shown in FIG. 2B and as suggested in FIG. 2C by the rendering in phantom on therapeutic face 20 of periphery 32 of medicament matrix 30.

Sandwiched between cover 56 and upper face 70 of substrate 18 is circuit board 68. On the side of circuit board 68 visible in FIG. 3B is a portion of a support face 76 thereof upon which are carried electronic circuitry 60, power source 62, and user switch 64. These and other electrical circuit elements of medicament matrix 30 are electrically interconnected by an electrically-conductive printed circuit 78 that is applied to support face 76, usually before other electrical circuit elements are mounted on circuit board 68. The depiction of printed circuit 78 in FIG. 3B and thereafter herein is entirely schematic and is not intended to reveal any details about the layout particulars of printed circuit 78.

Power source 62 is, by way of example, a miniature battery of about 3 volts potential. The current supplied by power source 34 to electronic circuitry 60 is thus non-alternating. Power source 62 may be a battery of higher or lower output potential, or power source 62 may be a plurality of series-connected batteries of equal or unequal output potential. Accordingly, for most medical applications, the output voltage produced by power source 62 ranges from about 1.00 volt to about 15.00 volts. Alternatively, the output voltage produced by power source 62 ranges from about 2.00 volts to about 9.00 volts, or from about 3.00 volts to about 6.00 volts.

In general, the greater the output voltage produced by a mobile power source, such as power source 62 associated with an active transdermal medicament patch, the larger will be the skin current I_{S} produced by that medicament patch, and the shorter will be the therapy period required to enable that medicament patch to administer any predetermined total dosage D_{T} of medicament. While such a result is salutary relative to minimizing the time during which a patient is required to be encumbered by wearing the medicament patch, the larger the skin current I_{S} produced by a medicament patch, the greater the likelihood that a wearer of the medicament patch will experience uncomfortable sensations, or even pain, during therapy. Accordingly, an unavoidable tradeoff exists between the desirable ends of comfort and of speedy therapy. Lower levels of power source output, such as those endorsed by teachings of the present invention, are calculated to increase patient comfort and to improve the likelihood that a patient will be willing to successfully complete a prescribed course of therapy, once that course of therapy has been undertaken.

Support face 76 of circuit board 68 has a complex periphery 80 that assumes an irregular, asymmetrical barbell-shape. Alternative configurations in circuit board 68 would not depart from the teachings of the present invention. At a first end 82 of circuit board 68 located in proximity to first electrode aperture 40, periphery 80 of support face 76 is similar in shape, but smaller in extent than first electrode aperture 40. At a second end 84 of circuit board 68 located in proximity to second electrode aperture 44, periphery 80 of support face 76 is similar in shape, but smaller in extent than second electrode aperture 48. Interconnecting first end 82 and second end 84 of circuit board 68 is an intermediate portion 86 of circuit board 68 in which periphery 80 of support face 76 is made up of linear segments.

Electronic circuitry 60 is mounted on support face 76 at first end 82 of circuit board 68. Power source 62 and user switch 64 are mounted on support face 74 of intermediate portion 86 of circuit board 68. Support face 76 at first end 82 of circuit board 68 is shown as being free of electrical circuit elements, other than printed circuit 78. The positions of such electrical circuit element's of medicament patch 16 may be altered without departing from the teachings of the present invention.

Superimposed by way of reference in phantom on upper face 70 of substrate 18 is periphery 80 of intermediate portion 86 of circuit board 68. In the assembled condition of medicament patch 16 shown in FIG. 3A, intermediate portion 86 extends longitudinally along substrate 18 between first electrode aperture 40 and second electrode aperture 48 and laterally thereof to a linear portion 90 of periphery 72 of upper face 70 of substrate 18. On upper face 70 of substrate 18, the phantom representation of intermediate portion 86 defines a circuit board contact area 88. In circuit board contact area 88 the side of circuit board 68 not visible in FIG. 3B engages and may thus be secured, as with adhesive, to upper face 70 of substrate 18.

Circuit board 68 is manufactured from an electrically-nonconductive material. Depending on the absolute size of circuit board 68 and the relative size of circuit board 68 to the size of substrate 18, the material from which circuit board 68 is fabricated can be rigid or minimally flexible. In the assembled condition of medicament patch 16, however, rigidity in circuit board 68 preferably does not prevent medicament patch 16 from being able to conform to curving skin surfaces at locations on the person of patient at which iontophoretic therapy is to be provided. The embodiment of circuit board 68 shown in FIG. 3B is manufactured from thin sheeting, such as sheeting made from a flexible polyester film, such as Mylar® brand polyester film manufactures by DuPont Teijin Films U.S. Ltd. of Hopewell, Va., U.S.A. As a result, circuit board 68 is relatively insubstantial and highly flexible.

Intermediate portion 86 of circuit board 68 includes a single layer of circuit board material. By contrast, as revealed in the enlarged portion of periphery 80 of support face 76 of first end 82 of circuit board 68 included in FIG. 3B, first end 82 of circuit board 68 includes a primary layer 92 above a substantially congruent secondary layer 94. Primary layer 92 of first end 82 of circuit board 68 carries electronic circuitry 60 and is a coplanar extension of intermediate portion 86. Similarly, as revealed in the enlarged portion of periphery 80 of support face 76 of second end 84 of circuit board 68 included in FIG. 3B, second end 84 of circuit board 68 includes a primary layer 96 above a substantially congruent secondary layer 98. Primary layer 96 of second end 84 of circuit board 68 carries a portion of printed circuit 78 and is also a coplanar extension of intermediate portion 86.

FIG. 3C is a perspective view of circuit board 68 of FIG. 3B. As indicated by arrow R₉₄₍₁₎ in FIG. 3C, secondary layer 94 of first end 82 of circuit board 68 has been rotated by 90 degrees in a clockwise direction out of the position thereof shown in FIG. 3B about a first axis A₁ located between secondary layer 94 and primary layer 92 of circuit board 68. In a somewhat similar manner, as indicated by arrow R₉₈₍₁₎ in FIG. 3C, secondary layer 98 of second end 84 of circuit board 68 has been rotated by 90 degrees in a counter clockwise direction out of the position thereof shown in FIG. 3B about a second axis A₂ located between secondary layer 98 and primary layer 96 of circuit board 68. First axis A₁ and second axis A₂ are generally parallel to one another and perpendicular to the longitudinal extent of circuit board 68 at the opposite ends thereof. Variations in such relationships would not be contrary to teachings of the present invention, as first axis A₁ and second axis A₂ can with substantially equivalent efficacy be intersecting relative to each other, or be individually or jointly located to one side or on opposite sides of the longitudinal extent of a circuit board, such as circuit board 68.

The partial disassembly of circuit board 68 depicted in FIG. 3C reveals that at first axis A₁, primary layer 92 and secondary layer 94 of first end 82 of circuit board 68 are connected by a bendable first electrode hinge 100. Similarly, at second axis A₂, primary layer 96 and secondary layer 98 of second end 84 of circuit board 68 are connected by a bendable second electrode hinge 102.

Either or both of first electrode hinge 100 and second electrode hinge 102 may be structures distinct from the portions of circuit board 68 interconnected thereby. In such an embodiment of a circuit board incorporating teachings of the present invention, one or both of secondary layer 94 and secondary layer 98 would be manufactured as distinct articles and then interconnected during further manufacturing activities by a corresponding one or both of first electrode hinge 100 and second electrode hinge 102. This could be a desirable arrangement, where the material of circuit board 68 is rigid or only partially flexible. Then, secondary layer 94, secondary layer 98, and the central portion of circuit board 68 between first axis A₁ and second axis A₂ could be manufactured from such a rigid or only partially flexible material and subsequently interconnected by flexible or mechanically bendable hinges, such as first electrode hinge 100 and second electrode hinge 102.

In the embodiment of circuit board 68 illustrated, however, first electrode hinge 100 and second electrode hinge 102 are coplanar extension of the portions of circuit board 68 interconnected thereby. The required capacity for bending in first electrode hinge 100 and second electrode hinge 102 arises from the flexibility of the material of which circuit board 68 is manufactured. Were that material rigid or only partially flexible, the degree of bendability required in first electrode hinge 100 and second electrode hinge 102 can be achieved without departing from teachings of the present invention by thinning or scoring the side of each of first electrode hinge 100 and second electrode hinge 102 that is not visible in FIG. 3C.

Thus, support face 76 of circuit board 68 extends in a continuous manner across first electrode hinge 100 to secondary layer 94 of first end 82 and across second electrode hinge 102 to secondary layer 98 of second end 84. Active electrode 50 can be appreciated from FIG. 3C to be carried on a portion of support face 76 that extends onto secondary layer 98 of second end 84 of circuit board 68 and to be electrically coupled to other electrical circuit elements of medicament patch 16 by the portion of printed circuit 78 that traverses second electrode hinge 102.

Correspondingly, the side of circuit board 68 opposite from support face 76 thereof is a continuous surface that may, if convenient, remain entirely free of electrical circuit elements. A portion of such a continuous attachment face 104 of circuit board 68 is visible on the side of secondary layer 94 of first end 82 of circuit board 68 presented in FIG. 3C. In the folded, compact state of circuit board 68 depicted earlier in FIG. 3C, attachment face 104 on secondary layer 94 of first end 82 of circuit board 68 engages attachment face 104 on primary layer 92 of first end 82, while attachment face 104 on secondary layer 98 of second end 84 engages attachment face 104 on primary layer 96 of second end 84. These relationships are depicted explicitly subsequently in FIG. 4.

FIG. 3D is a perspective view of circuit board 68 of FIG. 3C. As indicated by arrow R₉₄₍₂₎ in FIG. 3D, secondary layer 94 of first end 82 of circuit board 68 has been rotated by an additional 90 degrees in a clockwise direction out of the position thereof shown in FIG. 3C about first axis A₁. As indicated by arrow R₉₈₍₂₎ in FIG. 3D, secondary layer 98 of second end 84 of circuit board 68 has been rotated by an additional 90 degrees in a counter clockwise direction out of the position thereof shown in FIG. 3C about a second axis A₂. Thus, depicted in FIG. 3D is the fully unfolded, planar state of circuit board 68.

In view of the sequence of views of circuit board 68 presented in FIGS. 3B-3D, it is apparent that in one aspect of the present invention an active transdermal medicament patch employing a circuit board having mounted on an attachment face thereof a power source and an electrode, such as return electrode 42 or active electrode 50, is provided with electrode flexion means that traverses the circuit board intermediate the electrode and the power source for permitting bending of the circuit board between a planar state of the circuit board and a compact state of the circuit board. In the compact state of the circuit board, a portion of the attachment face in an electrode region of the circuit board located on the same side of the electrode flexion means as the electrode engages a portion of the attachment face in a power source region of the circuit board located on the same side of the electrode flexion means as the power source.

Pursuant to such teachings, it is possible in an active transdermal medicament patch to benefit from the use of a circuit board that is in effect electrically two-sided, but that carries only on a single side thereof the electrical circuit components of the medicament patch. This leaves the other side of the circuit board free of electrical circuit components. The freedom to maintain one side of the circuit board free of electrical circuit components is an optional benefit of an electrode flexion means incorporating teachings of the present invention.

As shown by way of example in FIG. 3D relative to first electrode hinge 100, circuit board 68 includes a first electrode region corresponding to secondary layer 94 of first end 82 and a power source region corresponding to the portion of circuit board 68 on the same side of first axis A₁ as power source 62. First electrode hinge 100 traverses circuit board 68 between return electrode 42 and power source 62 and permits circuit board 68 to bend out of the planar state thereof shown in FIG. 3D and into a more compact state thereof shown in FIG. 3B. In the compact state of circuit board 68, attachment face 104 on secondary layer 94 of first end 82 of circuit board 68 engages attachment face 104 on primary layer 92.

As shown by way of example in FIG. 3D relative to second electrode hinge 102, circuit board 68 includes a second electrode region corresponding to secondary layer 98 of second end 84 and a power source region corresponding to the portion of circuit board 68 on the same side of second axis A₂ as power source 62. Second electrode hinge 102 traverses circuit board 68 between active electrode 50 and power source 62 and permits circuit board 68 to bend out of the planar state thereof shown in FIG. 3D and into a more compact state thereof shown in FIG. 3B. In the compact state of circuit board 68, attachment face 104 on secondary layer 98 of first end 84 of circuit board 68 engages attachment face 104 on primary layer 96.

In FIG. 3D, return electrode 42 is depicted above and separated from support face 76 of circuit board 68. Revealed thereby is a return electrode contact pad 106 in which printed circuit 78 terminates on secondary layer 94 of first end 82 of circuit board 68. Superimposed by way of reference in phantom on support face 76 is periphery 44 of return electrode 42, which in the assembled condition of medicament patch 16 shown in FIG. 2B entirely obscures return electrode contact pad 106.

Active electrode 50 is depicted in FIG. 3D above and separated from support face 76 of circuit board 68. Revealed thereby is an active electrode contact pad 108 in which printed circuit 78 terminates on secondary layer 98 of second end 84 of circuit board 68. Superimposed by way of reference in phantom on support face 76 is periphery 106 of backing layer 52 of active electrode 50, which in the assembled condition of medicament patch 16 shown in FIG. 2B entirely obscures active electrode contact pad 108.

FIG. 4 is a cross-sectional elevation view of medicament patch 16 taken along section line 4-4 in FIG. 2A. As a result, FIG. 4 depicts in edge view both sides of substrate 18, as well as the interaction by way of first electrode aperture 40 and second electrode aperture 48 of other elements of medicament patch 16 discussed previously. In particular, circuit board 68 is shown in the fully folded, compact state thereof carrying electrical circuit components. From among the electrical circuit components carried on circuit board 68, printed circuit 78 been omitted out of convenience due to the thinness thereof. Nonetheless, the entirety of printed circuit 78 is disposed as shown in FIG. 3D, on support face 76 along with the balance of the electrical circuit elements of medicament patch 16.

As suggested by arrow S in FIG. 4, release liner 22 is in the process of being peeled from therapeutic face 20 of substrate 18, thereby to free the adhesive coating on therapeutic face 20 for the releasable attachment of medicament patch 16 to the skin of a patient. Simultaneously, the detachment of release liner 22 from medicament patch 16 will result in the removal of stray droplets of medicament solution 36. Securement surface 74 of medicament matrix 30 engages pH-control layer 54 and backing layer 52 of active electrode 50 interior of second electrode aperture 48. In second end 84 of circuit board 68, attachment face 104 of secondary layer 98 engages attachment face 104 of primary layer 96. Electronic circuitry 60, power source 62, and user switch 64 are carried on support face 76 of circuit board 68 and sealed therewith against upper face 70 of substrate 18 by cover 56. In first end 82 of circuit board 68, attachment face 104 of secondary layer 94 engages attachment face 104 of primary layer 92 interior of first electrode aperture 40

FIGS. 5A and 5B are related diagrams that compare the movement of medicaments of differing polarities through the skin of a wearer of medicament patch 16. The alterations in electrical interconnections required among element of medicament patch 16 to produce those movements are not illustrated, but will be mentioned.

FIG. 5A illustrates the movement of molecules of a positive medicament M⁺ that exhibits a net positive polarity. Therapeutic face 20 of substrate 18 is shown as being disposed against the surface 110 of skin 112. Then skin contact surface 34 of medicament matrix 30 and skin contact surface 46 of return electrode 42 each electrically conductively engage surface 110 of skin 112 at separated locations. Aside from the conductivity of skin 112, these locations are electrically isolated from each other. The negative pole of power source 34 is coupled directly or indirectly to return electrode 42. The positive pole of power source 62 is coupled directly or indirectly to medicament matrix 30, which engages skin 112 at a location remote from return electrode 42. The electromotive differential thusly applied to skin 112 between medicament matrix 30 and return electrode 42 induces molecules of positive medicament M⁺ to move as positive ions out of medicament matrix 30 toward skin 112, across the unbroken surface 110 of skin 112, and through skin 112 in the direction of return electrode 42. This movement is indicated in FIG. 5A by a dashed arrow labeled M⁺.

In electrical circuits, the flow of electrical current is conventionally indicated as a flow through the circuit from the positive to the negative pole of the power source employed therewith. Therefore, in FIG. 5A, an electrical skin current I_{S} is schematically indicated by a solid arrow to flow through skin 112 from medicament matrix 30, which is associated with the positive pole of power source 62, to return electrode 42 associated with the negative pole of power source 62. In the use of medicament patch 16 to administer a positive medicament M⁺, the direction of movement of molecules of positive medicament M⁺ through skin 112 thus coincides with the direction of skin current I_{S}.

While living tissue is a conductor of electric current, living tissue does nonetheless resist the flow of electrical current therethrough. It is the function of power source 62 to apply a sufficient electromotive force differential through skin 112 between medicament matrix 30 and return electrode 42 as to overcome this resistance. The presence of electrical resistance in skin 112 is indicated schematically in FIG. 5A as skin resistance R_{S}. Skin resistance R_{S} varies among human subjects over a wide range. Generally, within a few minutes of beginning to conduct a skin current, such as skin current I_{S}, the skin resistance R_{S} of most subjects undergoes transient changes and stabilizes at about 10 kilo-ohms, or more broadly stabilizes in a range of from about 10 kilo-ohms to about 50 kilo-ohms.

In FIG. 5B, the transcutaneous administration is depicted of molecules of a negative medicament M⁻ that exhibits a net negative polarity. Therapeutic face 20 of substrate 18 is shown again as being disposed against surface 110 of skin 112. Then skin contact surface 34 of medicament matrix 30 and skin contact surface 46 of return electrode 42 each electrically conductively engage surface 110 of skin 112 at separated locations. Aside from the conductivity of skin 112, these locations are electrically isolated from each other. The presence of electrical resistance in skin 112 is indicated schematically in FIG. 5B as skin resistance R_{S}.

To infuse a negative medicament M⁻, the electrical components of a medicament patch incorporating teachings of the present invention must be altered from those described above relative to FIG. 5A. Accordingly, the positive pole of power source 62 is coupled directly or indirectly to return electrode 42. Correspondingly, the negative pole of power source 62 is coupled directly or indirectly to medicament matrix 30. The electromotive differential thusly applied to skin 112 between return electrode 42 and medicament matrix 30 induces molecules of negative medicament M⁻ to move as negative ions out of medicament matrix 30 toward skin 112, across the unbroken surface 110 of skin 112, and through skin 112 in the direction of return electrode 42. This movement is indicated in FIG. 5B by a dashed arrow labeled M⁻.

The flow of electrical current in an electrical circuit is conventionally indicated as a flow through the circuit from the positive to the negative pole of the power source employed therewith. In FIG. 5B, a skin current I_{S} schematically indicated by a solid arrow to flow through skin 112 toward medicament matrix 30, which is associated with the negative pole of power source 62, from return electrode 42 associated with the positive pole of power source 62. In the use of medicament patch 16 to administer negative medicament M⁻, the movement of molecules of negative medicament M⁻ through skin 112 is in a direction that is opposite to that of skin current I_{S}.

For convenience and consistency in discussing various embodiments of the invention, the convention will be uniformly observed hereinafter that a negative medicament is to be administered. Nonetheless, this is not an indication that the teachings of the present invention have relevance exclusively to the administration of negative medicaments, as the present invention has applicability with equal efficacy to the administration of positive medicaments.

According to another aspect of the present invention, an active transdermal medicament patch, such as medicament patch 16 in FIGS. 1-5B, includes dosage control means non-removably carried on the substrate of the medicament patch for limiting to a predetermined medicament quantity, or dosage D_{T}, the total medicament administered into the skin of the patient by iontophoretic migration during a predetermined therapy period T_{M}. The dosage control means does so, notwithstanding transient electrical behaviors cause by various structures employed in a fully-integrated active transdermal medicament patch.

The inventive dosage control means is driven by a power source that is carried on a substrate shared therewith. Variability is, nonetheless, inherent in the output of a portable power source, like power source 62. Such a power source will exhibit a precipitous decline in output of at least 5% upon being first activated. Thereafter, the output of the power source will decline relatively steadily in output by about 5% or more during each succeeding hour of operation.

Similarly, certain electrical components of the types used in the inventive circuit disclosed herein are occasionally susceptible to mildly transient start-up performances, caused by heating or other factors. These transients stabilize after a relatively short fraction of any normal therapy period T_{M} and produce no more than a negligible effect on the overall dosage D_{T} of medicament ultimately administered during that entire therapy period.

In designing the inventive dosage control means, it has proven acceptable to assume that a power source of the type used with a fully integrated active transdermal medicament patch causes a substantially constant skin current I_{S} to flow through the medicament matrix of the medicament patch and skin of a wearer of the medicament patch during the entire course of therapy period T_{M}. In this manner, the total dosage D_{T} of medicament delivered by an active transdermal medicament patch incorporating teachings of the present invention is determinable with reasonable medical reliability by reference to the total of the time during which the medicament patch is employed for therapy.

As a result, it is contemplated that any such biological or electrical transients as might be observable in commencing the administration of medicament using apparatus and methods of the present invention do not derogate from what is medically accepted to be a substantially constant flow of skin current through the medicament matrix of an associated medicament patch and the skin of a wearer of the medicament patch during the entire course of some predetermined therapy period. This is the case, however, only once that skin current I_{S} has actually commenced.

Upon the initial disposition of the inventive active transdermal medicament patch against the skin of a patient, the resistance of the skin to the passage of electrical current therethrough is so high as to be considered an open circuit that precludes the passage of any skin current I_{S} whatsoever. At such occasions, the resistance of the skin to the passage of electrical current is far higher than any skin resistance R_{S} that permits a flow of skin current to be initiated and continued.

Thus, to initiate the administration of medicament, potentially extremely high initial skin resistances R_{S} must be overcome. Doing so under conditions that prevail with disposable iontophoresis patches, presents challenges. Often an extended period of minutes is required before any substantial skin current I_{S} can be induced. During this time, under the influence of the electrical potential applied by between the medicament matrix and the return electrode to the skin, the iontophoresis patch is developing and enlarging current pathways through the outer layers of skin into the underlying living dermis. Typically these initial current pathways develop first in sweat glands and in hair follicles. During this process, which is termed electroporation, skin resistance drops gradually. Eventually, a sustainable substantially steady state rate of skin current I_{S} flow commences.

Even upon establishing a skin current I_{S}, the skin resistance of most patients continues to undergo gradual transient changes before fully stabilizing. Accordingly, for a few initial minutes of a commenced predetermined therapy period T_{M}, the amount of skin current I_{S} that will flow through the skin will vary somewhat from the stable level subsequently observed during the balance of therapy period T_{M}. Nonetheless, over a full therapy period T_{M} of a few hours, these initial variations in the amount of skin current I_{S} caused by transients in skin resistance R_{S} have been determined to have a negligible effect on the overall dose of medicament ultimately administered.

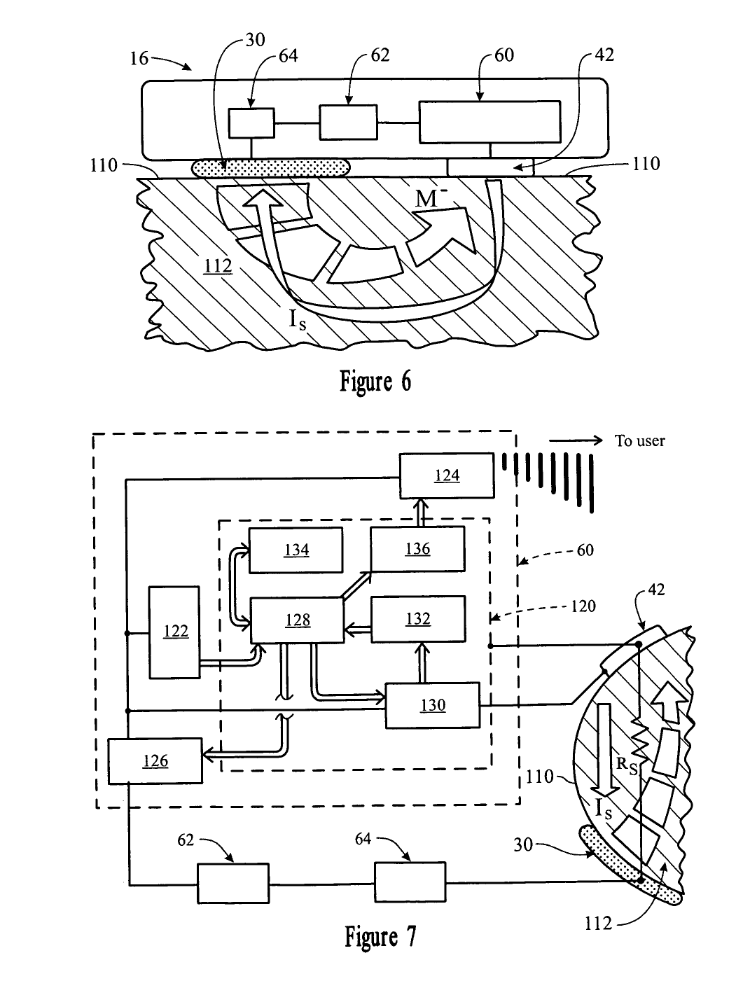

By way of example and not limitation, FIGS. 6 and 7 taken together depict medicament patch 16 carrying medicament matrix 30 and return electrode 42, each of which is in electrically conductive engagement with surface 110 of skin 112 of a patient. Skin current I_{S} has commenced, and the iontophoretic migration of negative medicament M⁻ is taking place. Therapy period T_{M} has begun. Thereafter, a medically acceptable substantially constant skin current flows through medicament matrix 30 and skin 112. Eventually, a total predetermined dosage D_{T} of medicament is delivered.

FIG. 7 in particular depicts various structural and functional components of electronic circuitry 60, including an embodiment of an inventive dosage control means. As shown by way of example and not limitation, a medicament migration monitor 120 coupled with power source 62 delivers electrical power to return electrode 42 on surface 110 of skin 112. Medicament migration monitor 120 periodically measures the rate of iontophoretic migration and correspondingly produces an output signal that is indicative of the status of that iontophoretic migration. A clock 122 receives power from power source 62 in the same manner as medicament migration monitor 120 and functions to communicate timing information to medicament migration monitor 120 continuously once user switch 64 has been activated. Also within electronic circuitry 60 and thus also carried non-removably on medicament patch 16 is dosing verification means for confirming to a user that iontophoretic migration is occurring. Also shown by way of example is an indicator circuit 124 that will be discussed in additional detail subsequently. Finally, a shutoff switch 126 is interposed between power source 62 and the elements of electronic circuitry 60 introduced above. Shutoff switch 126 is activatable by medicament migration monitor 120 to disable power source 62 and terminate the flow of skin current I_{S} and the iontophoretic migration of negative medicament M⁻ through skin 112.

A controller 128 supervises the operation of the other elements of medicament migration monitor 120, as well as the eventual activation of shutoff switch 126. Electrical power from power source 62 is delivered to return electrode 42 through a voltage sampler 130 that operates as directed by controller 128. Voltage sampler 130 produces an output signal reflecting the resistance to electrical current flow through skin 112 between medicament matrix 30 and return electrode 42. A signal comparator 132 evaluates the output signal from voltage sampler 130 and classifies the electrical current flow resistance among a predetermined typography of possible electrical current flow resistances having relevance to the status of the iontophoretic migration being induced by medicament patch 16. That predetermined typography includes: (a) an extremely elevated skin resistance R_{∞} reliably understandable as signifying the existence of an open circuit at the skin of the patient; (b) a high skin resistance reliably understandable as signifying the progress of skin electroporation; and (c) a normal skin resistance reliably understandable as signifying the existence of a closed circuit through skin 112 between medicament matrix 30 and return electrode 42. The output from signal comparator 132 is communicated to controller 128, which activates an indicator driver 136 corresponding, thereby to inform a user of the status of the iontophoretic migration of negative medicament M⁻. This is accomplished through the operation of indicator circuit 124. Once a normal skin resistance is detected by voltage sampler 130 and interpreted as such by signal comparator 132, controller 128 activates a dosage timer 134 that operates as long as the iontophoretic migration of negative medicament M⁻ continues. Dosage timer 134 continues in this manner, producing as an output signal a running cumulative total of the amount of negative medicament M⁻ delivered into skin 112 by iontophoretic migration.

Among the electrical interconnections presented in FIG. 7, power source 62 is so electrically coupled between medicament matrix 30 and return electrode 42 through skin 112 as to cause iontophoretic migration of negative medicament M⁻ to occur at a substantially constant rate. Then, controller 128 of medicament migration monitor 120 activates shutoff switch 126 only when the output signal of dosage timer 134 equals the ratio of predetermined medicament total dosage D_{T} divided by that substantially constant rate of iontophoretic migration.

In some instances, predetermined therapy period T_{M} is made up of a plurality of temporally non-contiguous therapy subsessions. Under such conditions, controller 128 of medicament migration monitor 120 may direct indicator driver 136 to operate indicator circuit 124 in a distinct delivery confirmation mode during each of the therapy subsessions, respectively.

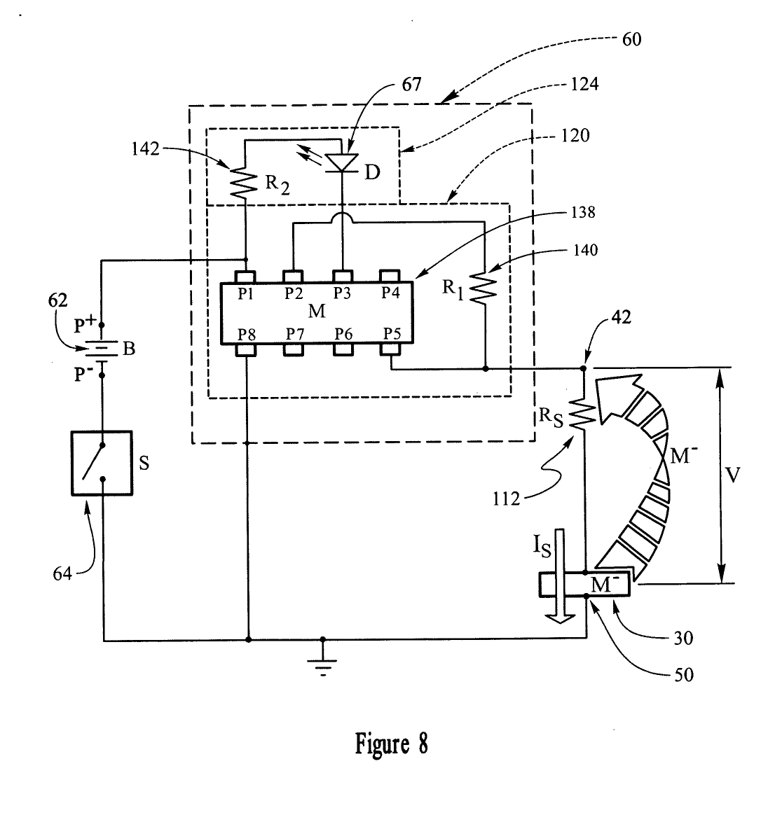

FIG. 8 is a more particular embodiment of electronic circuitry 60 that is capable of performing the functions of a dosage control means according to teachings of the present invention. There, medicament migration monitor 120 of electronic circuitry 60 is seen to be is coupled directly to the positive pole P⁺ of power source 62. Power source 62 then supplies a voltage that drives medicament migration monitor 120 and the other elements of electronic circuitry 60. The output of medicament migration monitor 120 is supplied to return electrode 42, which engages skin 112 of a patient. Together with power source 62, medicament migration monitor 120 in due course causes skin current I_{S} to flow through skin 112 from return electrode 42 in the direction shown, overcoming in the process electrical skin resistance R_{S} of skin 112.

The negative pole P⁻ of power source 62 is coupled through user switch 64 and active electrode 50 to medicament matrix 30, which engages skin 112 of a patient at a location that is remote from return electrode 42. According to the convention set forth earlier, medicament matrix 30 is filled with molecules of negative medicament M⁻. As a result, the electrical potential correspondingly imposed on skin 112 between return electrode 42 and medicament matrix 30, induces iontophoretic migration of molecules of negative medicament M⁻ from medicament matrix 30, through skin 112, and toward return electrode 42 in a direction that is opposite to that of skin current I_{S}.

Medicament migration monitor 120 includes a programmable microprocessor 138 having contact pins P1-P8. Microprocessor 138 is a semiconductor chip that includes a read-only memory that retains data when power to microprocessor 138 is terminated, but that can be electronically erased and reprogrammed without being removed from the circuit board upon which microprocessor 138 is mounted with other electrical circuit components. Advantageously, microprocessor 138 exhibits low power consumption requirements, which is in harmony with the use of a small, non-rechargeable mobile power source, such as power source 62.

Software installed in microprocessor 138 enables various of contact pins P1-P8 to performing multiple functions. The physical size of microprocessor 138 is accordingly small as compared with a microprocessor carrying only single-use contact pins, and the physical coupling of microprocessor 138 with other electrical circuit elements of electronic circuitry 60 necessitates fewer lead attachment soldering operations than would be the case using single-use contact pins. This reduces manufacturing costs and failures, as well as contributes to a desirably small footprint in microprocessor 138.

In medicament migration monitor 120 contact pin P6 and contact pin P7 of microprocessor 138 are not used. Positive pole P⁺ of power source 62 is coupled directly to contact pin P1, which therefore functions as an input contact for microprocessor 138. Contact pin P8 is grounded. The voltage output from medicament migration monitor 120 appears at contact pin P5 of microprocessor 138, which therefore, functions as an output contact for microprocessor 138. Contact pin P5 is coupled directly to return electrode 42. To insure that the voltage appearing at contact pin P5 is a substantially invariant voltage output, a sensing resistor 140 is electrically coupled between contact pin P5 and contact pin P2, which therefore functions as a current monitoring contact for microprocessor 138.

According to an aspect of the present invention mentioned earlier, an active transdermal medicament patch, such as medicament patch 16 in FIGS. 1-5B, includes activity indication means non-removably carried on the substrate of the medicament patch for communicating to a user that iontophoretic migration is under way. As shown by way of example in FIG. 6, electronic circuitry 60 also encompasses indicator circuit 124. Indicator circuit 124 in turn includes light-emitting diode 67 and a bias resistor 142 that are series-connected between contact pin P1 of microprocessor 138 and contact pin P3, which therefore functions as an activity indication contact for microprocessor 138.

Electronic circuitry 60 necessarily encompasses within microprocessor 138 an indicator driver, such as indicator driver 136 shown in FIG. 7. The indicator driver operates light-emitting diode 67 in any selected manner preferred by medical personal and suited to the sensory capacities of the patient with whom medicament patch 16 is to be used for therapy. For example, indicator driver 136 shown in FIG. 7 might be directed by controller 128 to operate light-emitting diode 67 only on an intermittent basis during any therapy period in order to conserve the capacity of power source 62 for use by other elements of electronic circuitry 60.

The operation of light-emitting diode 67 by microprocessor 138 affords a visual indication that medicament migration monitor 120 is functioning. In the alternative, indicator circuit 124 could employ in place of light-emitting diode 67 an auditory indicator or a tactile indicator that engages skin 112 of the patient or that can be encountered at will by attending medical personnel in the manner of taking a pulse. Such a tactile indicator could, for example, be a vibrating element or a heating element. Auditory or tactile indicators may consume the output capacity of power source 62 more rapidly than a light-emitting diode, and particularly more rapidly than an intermittently-operated light-emitting diode.

The migration of medicament through skin 112 is reflected as a flow of skin current I_{S} from contact pin P5 of microprocessor 138 to return electrode 42. The flow of skin current I_{S} is detected at contact pin P2 of microprocessor 138, whereby microprocessor 138 is able over time, informed for example by dosage timer 134 shown in FIG. 7, to develop something analogous to a running cumulative total of the amount of medicament administered. When that running cumulative total reaches predetermined total dosage D_{T} of medicament, microprocessor 138 is programmed to function as a shutoff switch and disable power source 62, thereby terminating skin current I_{S} and the migration of medicament through skin 112.

The voltage V applied through skin 112 between return electrode 42 and medicament matrix 30 is maintained at a substantially invariant level for the full duration of a predetermined therapy period T_{M} that ranges in duration from about 1 hour to about 6 hours, or more narrowly from about 2 hours to about 4 hours. The voltage applied through skin 112 between return electrode 42 and medicament matrix 30 causes iontophoretic medicament migration to occur through skin 112 from medicament matrix 30 to return electrode 42 at a substantially constant rate.

When medicament migration occurs at a substantially constant rate, skin current I_{S} is substantially constant, and the integration function to be performed by microprocessor 138 in monitoring the administration of total dosage D_{T} of medicament is reduced to one of using clock 122 in microprocessor 138 to time the duration of the period during which the substantially constant skin current I_{S} has been produced. When the output of clock 122 reaches the ratio of total dosage D_{T} of medicament divided by the substantially constant skin current I_{S} that is supplied by power source 62, microprocessor 138 is programmed to function as a shutoff switch and disable power source 62, thereby terminating skin current I_{S} and the migration of additional medicament through skin 112.

For a skin resistance R_{S}=10 kilo-ohms, the following electrical circuit component values and identities in medicament migration monitor 120 and in indicator circuit 124 produced a substantially invariant voltage V=2.75 volts and a corresponding substantially constant skin current I_{S}=0.275 milliamperes during the course of a therapy period T_{M}=280 minutes:
M=8-pin, 8-bit flash microcontroller PIC 12 F 510-I/SN of the type manufactured by Microchip Technology Inc. of Chandler, Ariz. U.S.A;
D=green light-emitting diode PG 1112 H-TR of the type manufactured by Stanley Electric U.S. Co., Inc. of London, Ohio, U.S.A.;
B=3.0 volt lithium-manganese button cell CR 1025 of the type manufactured by Blueline Electronics Technology Co., Inc. of Hong Kong, R.O.C.;
R₁=100 kilo-ohm resistor ERJ-6 GEYJ 104 V of the type manufactured by Panasonic Corporation of North America of Secaucus, N.J. U.S.A.;
R₂=300 ohm printed resistor; and
S=pull tab switch fabricated from same polyester film as circuit board 68.
   Performance curves for such a medicament migration monitor 120 and such an indicator circuit 124 are included by way of example among the drawings.

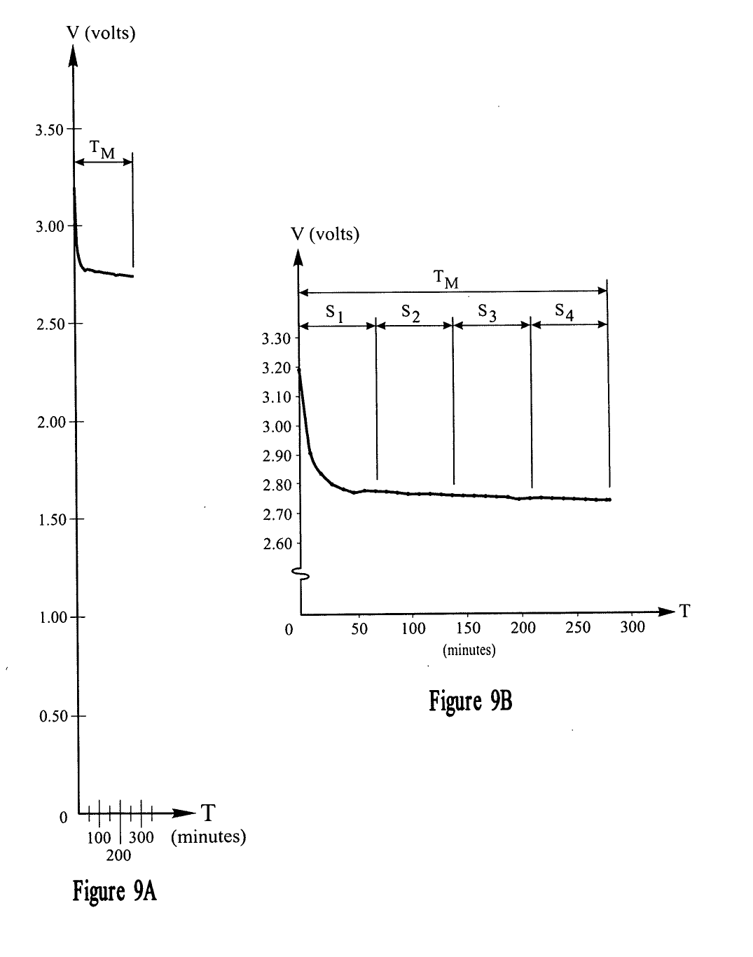

FIGS. 9A and 9B are the same performance curve, but drawn in contrasting respective scales to depict the voltage V applied by medicament migration monitor 120 across a skin resistance R_{S}=10 kilo-ohms over a predetermined therapy period T_{M}=280 minutes. In FIG. 9B, the enlarged-scale version of the voltage performance curve, therapy period T_{M} is for convenience of analysis divided into a plurality of four (4) equal therapy subsessions S₁, S₂, S₃, and S₄ of 70 minutes each.

Power source 62 is activated by a user through the operation of user switch 64. Initially, skin resistance R_{S} equals R_{∞}, and no skin current I_{S} flows. Gradually through the process of electroporation, skin resistance R_{S} is reduced. When skin resistance R_{S} reaches a value of skin resistance R_{N} at which skin current I_{S} begins to be able to flow, the timing of the administration of medicament begins. Only then is time set to T=0. Momentarily, voltage V=3.18 volts, greater even than the nominal 3.00 volt rating of power source 62 when configured as a battery B of the type specified in the above list of electrical circuit component in FIG. 8. From time T=0 minutes, voltage V declines steeply in a seemingly linear manner. By time T=5 minutes, voltage V=3.00 volts. Then, voltage V commences a relatively sharp decline in slope, decaying asymptotically toward the horizontal. At about time T=20 minutes, voltage V arrives at a substantially invariant voltage V=2.75±0.02 volts, which is then sustained throughout the balance of therapy subsession S₁ and all of therapy subsessions S₂, S₃, and S₄ remaining in therapy period T_{M}.

The initial behavior of voltage V depicted in FIGS. 9A and 9B at the commencement of therapy period T_{M} results from mildly transient start-up performances on the part of power source 62 and the electrical components of medicament migration monitor 120 and indicator circuit 124. Nonetheless, as will be observed subsequently, in the context of the totality of therapy period T_{M}, that initial transient behavior of voltage V has a negligible effect on the total dosage D_{T} of medicament administered.

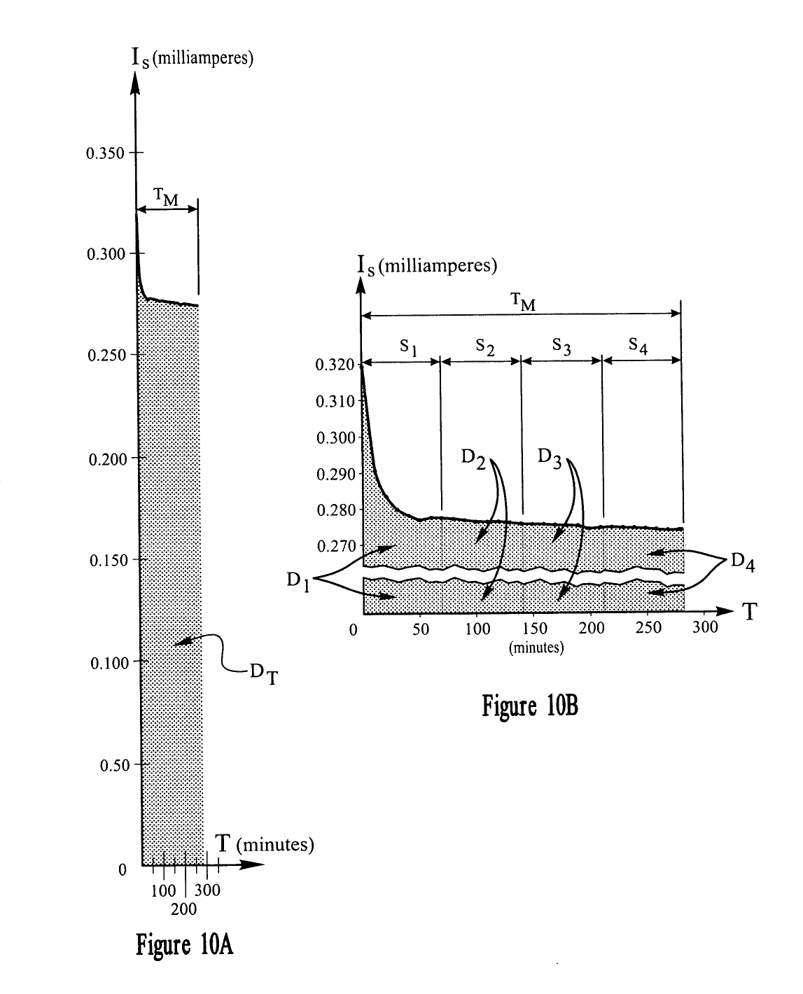

FIGS. 10A and 108B are the same performance curve, but drawn in contrasting respective scales to depict the skin current I_{S} produced by voltage V depicted in FIGS. 9A and 9B. In FIG. 10B, the enlarged-scale version of the skin current performance curve, therapy period T_{M} has for consistency of analysis been divided into the same plurality of therapy subsessions S₁, S₂, S₃, and S₄ as appeared in FIG. 9B.

The initial transient behavior of voltage V is closely reflected in skin current I_{S}.

At time T=0 minutes, skin current I_{S}=0.318 milliamperes. From time T=0 minutes, skin current I_{S} declines steeply in a seemingly linear manner. By time T=5 minutes, skin current I_{S}=0.300 milliamperes. Then, skin current I_{S} commences a relatively sharp decline in slope, decaying asymptotically toward the horizontal. At about time T=20 minutes, skin current I_{S} arrives at a substantially constant skin current I_{S}=0.275±0.02 milliamperes, which is then sustained throughout the balance of therapy subsession S₁ and all of therapy subsessions S₂, S₃, and S₄ remaining in therapy period T_{M}. In the context of the totality of therapy period T_{M}, that initial transient behavior of skin current I_{S} has a negligible effect on the total dosage D_{T} of medicament administered.

The area below the performance curve of skin current I_{S} in FIGS. 10A and 10B from time T=0 minutes until any given time T during therapy period T_{M} is equal to the cumulative dosage D of medicament administered through that time T. Thus, in FIG. 10A the area beneath the performance curve of skin current I_{S} between time T=0 minutes and time T=280 minutes at the conclusion of therapy period T_{M} is identified as the total dosage D_{T} of medicament administered. To facilitate continued analysis, in FIG. 10B the total dosage D_{T} of medicament administered has been divided into a plurality of four (4) medicament subdoses D₁, D₂, D₃, and D₄, which correspond in a one-to-one manner to the amount of medicament administered during each of therapy subsessions S₁, S₂, S₃, and S₄, respectively. Thus, therapy subdose D₁ represents the amount of medicament administered in therapy subsession S₁; therapy subdose D₂ represents the amount of medicament administered in therapy subsession S₂; and so forth.

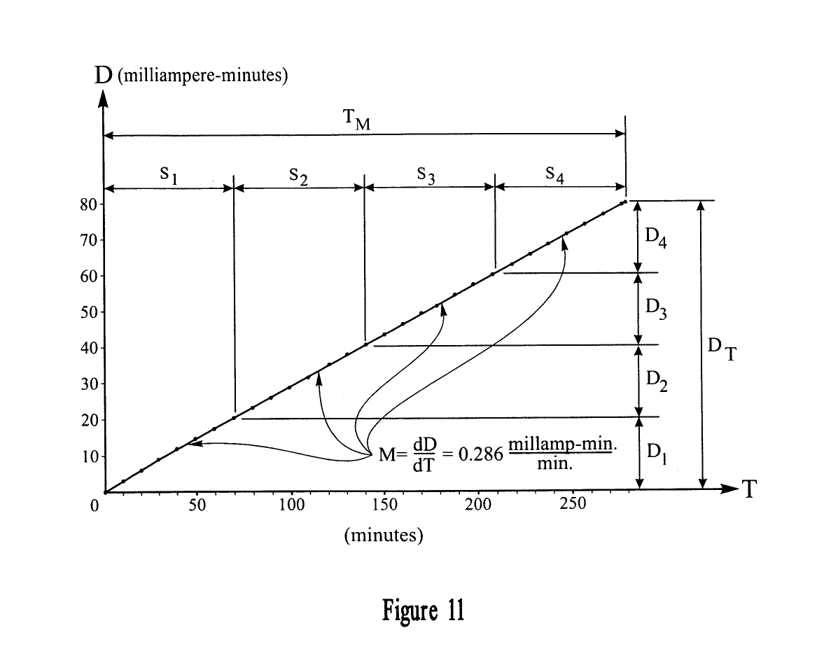

FIG. 11 is a performance curve showing the cumulative dosage D of medicament administered as a result of the imposition of the voltage V of FIGS. 7A-7B across a skin resistance R_{S}=10 kilo-ohms from time T=0 minutes at the start of therapy period T_{M} until the end of therapy period T_{M} at time T=280 minutes. The performance curve of FIG. 11 is thus derived directly from FIGS. 10A and 10B, being a plot of the value of the area beneath the performance curve of skin current I_{S} in those drawings. As can be observed, cumulative dosage D is substantially strictly linear, reflecting the administration in each of therapy subsessions S₁, S₂, S₃, and S₄ of corresponding equal medicament subdoses D₁, D₂, D₃, and D₄ of about 40 milliampere-minutes. Thus, during the entirety of therapy period T_{M}, the circuitry of FIG. 8 administers a total dosage D_{T}=280 milliampere-minutes of medicament at a substantially constant rate of about 0.286 milliampere-minutes per minute, the slope M of the performance curve of cumulative dosage D presented in FIG. 11.

During the administration of a medication using an active medicament patch, such as medicament patch 16, it may become necessary or it may occur accidentally that therapy is interrupted before the end of a full predetermined therapy period T_{M} during which a corresponding predetermined total dosage D_{T} of medicament was intended to be administered. This might occur, for example, due to the removal of medicament patch 16 from the skin of the patient. Once the interruption of therapy is detected, and the cause of the interruption remedied, therapy can and should be resumed toward the completion of the administration of total dosage D_{T} of medicament. Under such circumstances, uncertainty will exist relative to how much medicament was actually administered before the interruption. Correspondingly uncertain will be the amount of additional medicament that needs to be administered once therapy is resumed in order to cumulatively administer total dosage D_{T} of medicament.

Accordingly, in one aspect of the present invention, an active medicament patch, such as medicament patch 16, is provided with dosage control means carried non-removably on the substrate of the medicament patch for limiting to a predetermined medicament quantity the total medicament migrated iontophoretically from the medicament matrix into the skin of the patient during, perhaps, a plurality of temporally non-contiguous therapy subsessions. The portion of therapy period T_{M} preceding any interruption thereof and the balance of therapy period T_{M} that must of necessity be undertaken following such an interruption are examples of a pair of such temporally non-contiguous therapy subsessions.

Yet, it is contemplated that a dosage control means incorporating teachings of the present invention be able to accommodate for any number of interruptions in therapy during any single intended therapy period T_{M}. Such a situation might arise, for example, were it desirable under circumstances like those depicted in the performance curves of FIGS. 9A-11 to interrupt therapy for a brief respite at the end of several or each of therapy subsessions S₁, S₂, and S₃. Such an interruption or interruptions might be needed in order to inspect the skin of the patient at the site of therapy or to adjust the positioning of medicament patch 16 on the skin of the patient.

Accordingly, as shown by way of example in FIG. 8, a dosage control means incorporating teachings of the present invention includes medicament migration detector 120 that includes microprocessor 138 and sensing resistor 140 electrically coupled as shown to power source 62, return electrode 42, and medicament matrix 30. Medicament migration detector 120 continuously monitors the flow of skin current I_{S} and, thereby, the iontophoretic migration of medicament from medicament matrix 30 into the skin of the patient. As an output, medicament migration detector 120 through indicator circuit 124 continuously informs a user of the status of that iontophoretic medicament migration.

A dosage control means incorporating teachings of the present invention may, for example, be effected in the software in microprocessor 138, or in the alternative may be embodied in software or hardware located elsewhere than within microprocessor 138. A shutoff switch is used to disable power source 62 at a time from the initiation of iontophoretic migration corresponding to full predetermined therapy period T_{M}. Such a shutoff switch may, for example, be effected in the software in microprocessor 138, or in the alternative may be embodied in software or hardware located elsewhere than within microprocessor 138. In this manner, following any interruption in the administration of medication, the dosage control means resumes monitoring the amount of medication administered where that administration was at the time of the interruption.

Power source 62 may be so electrically coupled between return electrode 42 and medicament matrix 30 as to cause iontophoretic medicament migration from medicament matrix 30 into the skin of the patient to occur at a substantially constant rate. Under such circumstances, a dosage control means incorporating teachings of the present invention includes a medicament migration detector as described above and a dosage timer active only when the output of the medicament migration detector exceeds a predetermined minimum rate of medicament migration associated with a closed circuit. Such a dosage timer may, for example, be effected in the software in microprocessor 138, or in the alternative may be embodied in software or hardware located elsewhere than within microprocessor 138. A shutoff switch disables power source 62, when the duration of the activity of the dosage timer equals the ratio of the predetermined total dose D_{T} of medicament divided by the substantially constant rate of iontophoretic medicament migration being produced

It has been found to be helpful to apprise a user of an active medicament patch, such as medicament patch 16, as to the degree to which the administration of any total dosage D_{T} of medicament has been completed. Accordingly, in another aspect of the present invention, an active medicament patch, such as medicament patch 16, includes therapy status advisement means that is non-removably carried on the substrate of that medicament patch, and that is driven by a power source, such as power source 62. The therapy status advisement means performs the function of communicating to a user the extent of completion of predetermined therapy period T_{M} during which a medicament is to be iontophoretically delivered from medicament matrix 30 into the skin of a patient.

Accordingly, as shown by way of example in FIG. 8, a therapy status advisement means incorporating teachings of the present invention includes microprocessor 138, light-emitting diode 67, and bias resistor 132 as shown electrically coupled to power source 62, to return electrode 42, and to medicament matrix 30. In the alternative to a visual indicator, such as light-emitting diode 67, the therapy status advisement means may employ an auditory indicator or a tactile indicator of the type described earlier. The therapy status advisement means need not necessarily be contained within or associated with circuitry that, like medicament migration monitor 120, is capable of imposing a substantially invariant voltage V between return electrode 42 and medicament matrix 30.

Also included in a therapy status advisement means configured according to teachings of the present invention is a timer that is active only during therapy period T_{M} and a driver for light-emitting diode 67 that causes light-emitting diode 67 to operate only when the dosage timer is active, during perhaps various of a plurality of therapy subsessions. Typically, light-emitting diode 67 is operated intermittently to minimize power consumption. Such a timer and such a driver may, for example, be effected in the software in microprocessor 138, or in the alternative may be embodied in software or hardware located elsewhere than within microprocessor 138.

Therapy period T_{M} may include a sequence of non-overlapping predetermined therapy subsessions, such as therapy subsessions S₁, S₂, S₃, and S₄ of therapy period T_{M} depicted in the performance curves of FIGS. 9B, 10B, and 11. Therapy period T_{M} may include more or fewer therapy subsessions, and those therapy subsessions need not be of substantially equal duration, as in the case of therapy subsessions S₁, S₂, S₃, and S₄. Advantageously, the driver of the therapy status advisement means may then activate light-emitting diode 67, or any auditory or tactile indicator used in place thereof, in a distinct mode of operation during each of the therapy subsessions, respectively. Alternative or in addition thereto, the driver of the therapy status advisement means may cause light-emitting diode 67 or any auditory or tactile indicator used in place thereof, to operate in a contrasting transition mode at the end of a selected one or a selected plurality of the therapy subsessions, including at the end of final therapy subsession S₄ at the termination of therapy period T_{M}. Finally, the driver of the therapy status advisement means may cause light-emitting diode 67 or any auditory or tactile indicator used in place thereof, to operate in a contrasting alarm mode when the timer of the therapy status advisement means is deactivated prior to the termination of therapy period T_{M}. Such would be the case where therapy during a full predetermined therapy period T_{M} is interrupted due to the temporary removal of medicament patch 16 from the skin of the patient.

It is important during the initiation of operation that a user be advised accurately of the status of patch operation. Accordingly, the indicator electronic circuitry 60 initially gives indications that no current is flowing when the monitor thereof detects voltages corresponding to skin resistances in excess of an arbitrary upper threshold skin resistance R_{∞}, such as 3.0, 5.0, or even 10.0 MΩ. At this stage of operation skin current I_{S} so inconsequential as to be considered characteristic of an open circuit at the skin of the patient.

Once the presence of the iontophoresis patch on the skin has reduced skin resistance R_{S} to a value less than upper threshold skin resistance R_{∞}, the indicator of electronic circuitry 60 emits a distinct signal that is intended to advise a user that an initial transitional period of high skin resistance operation has commenced. During this high resistance mode of operation, skin current I_{S} is still taken to be negligible. High resistance operation continues until detected skin resistance R_{S} drops to or below a predetermined threshold value R_{N} that is equal to a predetermined percentage P of upper threshold skin resistance R_{∞}, such as 50, 25, 10, or even 1 percent of upper threshold skin resistance R_{∞}. Thereupon, steady state operation is considered to commence. The actual delivery of medicament ensues, and any involved dosage timer is started. The indicator emits another distinct signal that is informs a user that steady state operation is in progress.

The overall operation of therapy status advisement means is thus governed by a driver that activates light-emitting diode 67, or any auditory or tactile indicator used in place thereof, in a discrete variety of operative modes P, each of which is reflective of a foreseeable medicament administration status condition X. Each status condition X thus includes temporal and electrical information, information relative to the time T within therapy period T_{M} and information relative to the existence or nonexistence of skin current I_{S} in the skin of the patient. Temporally, status condition X can denote that therapy is in a specific one of a plurality of therapy subsessions, such as therapy subsessions S₁, S₂, S₃, and S₄, or that therapy is at the end of a chosen one or of all of those therapy subsessions. Electrically, status condition X denotes whether skin current I_{S} is flowing, or whether skin current I_{S} is zero by being less than some predetermined minimum amount chosen to evidence an open circuit. The later would be the case, for example, were the resistance between medicament matrix 30 and return electrode 42 to be detectable as resistance R_{∞}, a predetermined skin resistance at and above which an open circuit effectively exits in skin 112. Resistance R_{∞} thus well exceeds an arbitrary upper threshold beyond the range of the likely skin resistance R_{S} in any patient.

Relatedly, another predetermined threshold relevant to desirable operation of medicament patch 16 is resistance R_{N}, the upper limit of resistance R_{S} considered to be a closed circuit. Typically, resistance R_{N} is some predetermined percentage P of resistance R_{∞}. Above resistance R_{N}, but below resistance R_{∞}, a high resistance mode of operation is identified in which skin current I_{S} continues to be negligible, but during which the progress of electroporation is apparent.

In this light, the operative mode P of light-emitting diode 67, or any auditory or tactile indicator used in place thereof, is a function of status condition X. Presented below is a table listing typical status conditions X and an exemplary operative mode P(X) corresponding to each for a therapy period T_{M} that is comprised of a non-overlapping sequence of therapy subsessions S₁, S₂, S₃, and S₄. An operative open circuit mode is produced in light-emitting diode 67, whenever skin current skin resistance R_{S} is equal to resistance R_{∞}. Distinct first and second operative transition modes are produced in light-emitting diode 67 half way through therapy period T_{M} at the end of therapy subsession S₂, and at the completion of therapy period T_{M} when therapy subsession S₄ ends.

| Status condition X | Operative mode P(X) |
|---|---|
| S₁ | One (1) LED-flash of duration A₁ at regular |
| | intervals of duration E₁ |
| S₂ | Two (2) LED-flashes of duration A₁ at regular |
| | intervals of duration E₁ |
| S₃ | Three (3) LED-flashes of duration A₁ at |
| | regular intervals of duration E₁ |
| S₄ | Four (4) LED-flashes of duration A₁ at regular |
| | intervals of duration E₁ |
| R_{S} = R_{∞} | Continuous patterned LED-flashes at regular |
| (open circuit mode) | intervals of duration E₂ >> E₁, each |
| | pattern including an LED-flash of |
| | duration A₁, an interval of duration E₁, |
| | and an LED-flash of duration A₂ |
| R_{∞} > R_{S} ≧ R_{N} | Five (5) LED-plashes of duration A₁ at regular |
| (high resistance mode) | intervals of duration E₁ |
| S₂ has ended | Continuous LED-flashes of duration A₁ at |
| (first transition mode) | regular intervals of duration E₃ for |
| | an extended period of duration K₁ |
| T = T_{M} and S₂ has ended | Continuous LED-flashes of duration A₁ at |
| (second transition mode) | regular intervals of duration E₃ for |
| | an extended period of duration K₂ |

Typical possible durations for the events appearing among the operative modes P(X) in the table above are as follows:
A₁=0.25 seconds;
A₂=1.00 seconds;
E₁=0.50 seconds;
E₂=10.0 seconds;
E₃=5.0 seconds;
K₁=120 seconds; and
K₂=240 seconds.

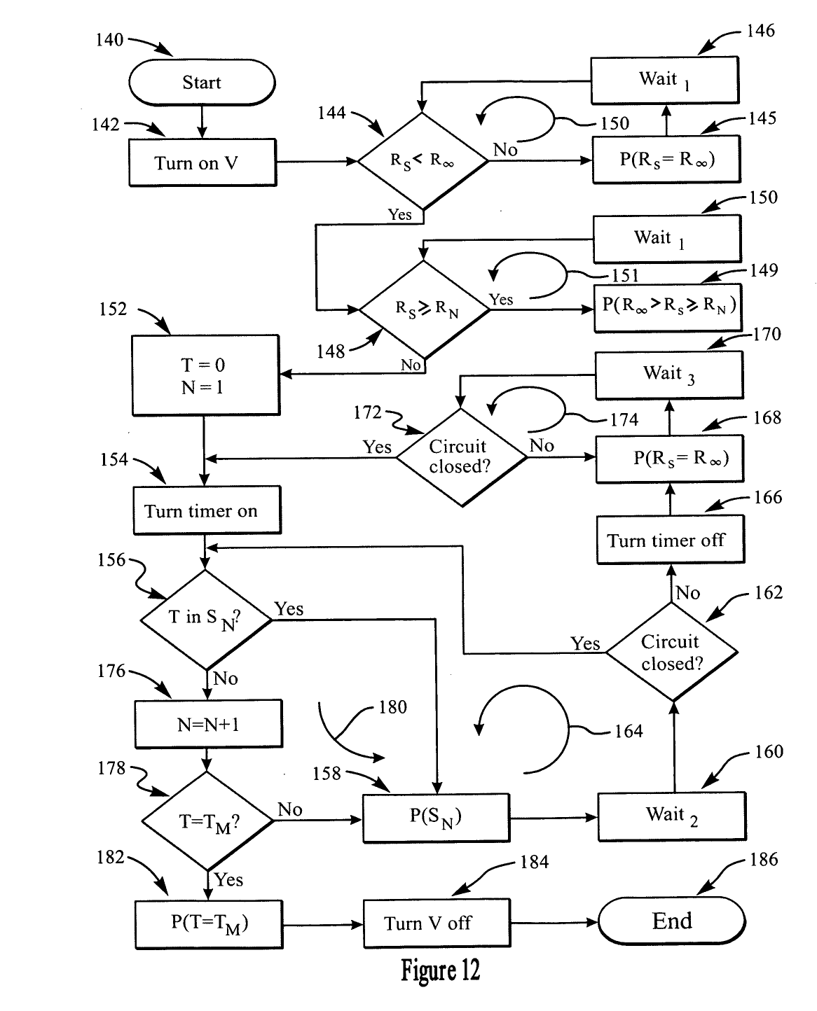

FIG. 12 is a flowchart of method steps involved in implementing operative mode P(X) as listed in the table above for all status conditions X, other than X=“S₂ has ended.” The activities required to implement operative mode P(S₂ has ended) have been omitted in FIG. 10 only to avoid redundancy. All of the method steps illustrated may be conducted, by way of example, by software in microprocessor 138 in FIG. 6, or in the alternative by software or hardware located elsewhere.

The depicted methodology commences at initiation oval 140 by turning voltage V on as required in procedure rectangle 142. This occurs when power source 62 is activated by a user through the operation of user switch 64. Thereupon, if medicament patch 16 is in place on skin 112 of a patient, medicament migration monitor 120 should begin to apply voltage V across skin 112 between medicament matrix 30 and return electrode 42, and in due course as a result of electroporation, skin current I_{S} should begin to flow.

These actions may not always succeed in creating a closed circuit in which a flow of skin current I_{S} possible. Accordingly, as required by decision diamond 144, microprocessor 138 inquires toward that end. If as a result, microprocessor 138 determines that no skin current I_{S} is flowing, because R_{S}<R_{∞}, then as stipulated in procedure rectangle 145, in order to alert a user that medicament patch 16 is not yet operating as intended, the driver of light-emitting diode 67 in microprocessor 138 operates light-emitting diode 67 in operative mode P(R_{S}=R_{∞}), the operative open circuit mode. As specified in procedure rectangle 149, microprocessor 138 then idles for a predetermined period Wait₁ during which to permit a user to detect and, if appropriate, to remedy the situation as when medicament patch is not affixed to the skin. After idling for predetermined period Wait₁, microprocessor 138 again undertakes the inquiry in decision diamond 144 to determine whether R_{S}<R_{∞} so that skin electroporation can be deemed to be progressing. If not, microprocessor 138 continues repeatedly to operate in a functional loop 147 that includes decision diamond 144, procedure rectangle 145, and procedure rectangle 146.

On any circuit of functional loop 151, if microprocessor 138 detects that skin electroporation is in progress, because R_{S}<R_{∞} then as required by decision diamond 148, microprocessor 138 inquires as to the progress of electroporation. If as a result, microprocessor 138 determines that skin electroporation is in progress but still R_{S}≧R_{∞}, then as stipulated in procedure rectangle 149, in order to alert a user that medicament patch 16 is beginning to cause electroporation as intended, the driver of light-emitting diode 67 in microprocessor 138 operates light-emitting diode 67 in operative mode P(R_{∞}>R_{S}≧R_{N}), the high resistance mode. As specified in procedure rectangle 149, microprocessor 138 then idles for a predetermined period Wait₁, and microprocessor 138 again undertakes the inquiry in decision diamond 148 to determine whether skin electroporation has completed sufficiently that R_{S} has become less than R_{N}. If not, microprocessor 138 continues repeatedly to operate in a functional loop 151 that includes decision diamond 148, procedure rectangle 149, and procedure rectangle 150.

On any circuit of functional loop 157, if microprocessor 138 detects that skin current I_{S} has commenced through skin 112, because R_{S} has become less than R_{N}, the depicted methodology moves ahead to procedure rectangle 152. Consequently, a timer in microprocessor 138 of the duration of therapy is prepared for activity by setting time T=0, and a counter N identifying the therapy subsession S_{N} in which therapy is occurring is set to N=1. This signifies that therapy subsession S₁ will be the initial therapy subsession. As directed in procedure rectangle 154, the timer in microprocessor 138 is turned on, and time T advances continuously from time T=0 until the timer is turned off.

In decision diamond 156, microprocessor 138 compares the ongoing time T to a schedule of times for the intended therapy subsessions to verify that therapy is occurring in therapy subsession S_{N} with N=1. If as a result, it is determined that that therapy is occurring in therapy subsession S₁, then as specified in procedure rectangle 158, the driver of light-emitting diode 67 in microprocessor 138 operates light-emitting diode 67 in operative mode P(S₁) to advise the user that medicament patch 16 is operational and that therapy is progressing in therapy subsession S₁. According to the above table of operative mode P(X), during therapy subsession S₁ light-emitting diode 67 is made to flash once for 0.25 seconds at regular intervals of 0.50 seconds.

In procedure rectangle 160, microprocessor 138 idles for a predetermined period Wait₂ and then undertakes the inquiry in decision diamond 162 to determine whether a closed circuit continues to exist in which a flow of skin current I_{S} is occurring. If it is determined that skin current I_{S} continues to be flowing, activity returns to decision diamond 156 and continues repeatedly through a functional loop 164 that includes decision diamond 156, procedure rectangle 158, procedure rectangle 160, and decision diamond 162.

On any transit of functional loop 164, if it is determined in decision diamond 162 that no skin current I_{S} is flowing, the timer in microprocessor 138 is turned off as required in procedure rectangle 166. Time T ceases to advance, until the timer is next turned on. As stipulated in procedure rectangle 168, in order to alert the user that medicament patch 16 is no longer operating as intended, the driver of light-emitting diode 67 in microprocessor 138 operates light-emitting diode 67 in operative mode P(R_{S}=R_{∞}), the operative open circuit mode. Then, as required in procedure rectangle 170, microprocessor 138 idles for a predetermined period Wait₃ to allow a user to detect and remedy the situation. After idling for predetermined period Wait₃, microprocessor 138 undertakes the inquiry in decision diamond 172 to determine whether skin current I_{S} has resumed. If not, microprocessor 138 continues repeatedly to operate in a functional loop 174 that includes decision diamond 172, procedure rectangle 168, and procedure rectangle 170.

On any transit of functional loop 174, if microprocessor 138 detects at decision diamond 172 that skin current I_{S} has recommenced through skin 112, the depicted methodology leaves functional loop 174 and moves ahead to procedure rectangle 154. The timer in microprocessor 138 is again turned on. As a consequence thereof, time T advances continuously once again, but from the time T at which the timer was turned off in procedure rectangle 166. Activity returns to functional loop 164, until such time as in undertaking the inquiry in decision diamond 156, microprocessor 138 compares time T to the schedule of times for the intended therapy subsessions and discovers that therapy is no longer in therapy subsession S_{N} with N=1.

Thereupon, the illustrated methodology advances to procedure rectangle 176, and microprocessor 138 increases counter N by one; so that N=2. As a consequence, therapy is understood to be starting the next successive therapy subsession S_{N+}, or in other words to be starting therapy subsession S₂, which follows therapy subsession S₁. In decision diamond 178, microprocessor 138 ascertains whether therapy period T_{M} has yet fully transpired. If not, the administration of total dosage D_{T} of medicament has not yet been completed, and the illustrated methodology returns to functional loop 164 by way of procedure rectangle 158, but with N=2. Procedure rectangle 176 and decision diamond 178 thus make up a functional branch 180 by which microprocessor 138 resisters that therapy has advanced into a successive therapy subsession.

On each successive circuit of functional loop 164, the driver of light-emitting diode 67 in microprocessor 138 operates light-emitting diode 67 in operative mode P(S₂) to advise the user that medicament patch 16 is operational and that therapy is progressing in therapy subsession S₂. According to the above table of operative mode P(X), during therapy subsession S₂ light-emitting diode 67 is made to flash twice for 0.25 seconds at regular intervals of 0.50 seconds. The illustrated methodology continues in functional loop 164, until the inquiry undertaken by microprocessor 138 in decision diamond 156 reveals that therapy subsession S₂ has been completed.

Then, by way of a functional branch 180 counter N is again increased by one, and activity resumes, reentering functional loop 164 through procedure rectangle 158. On each occasion that the inquiry in decision diamond 156 diverts activity out of functional loop 164 and through functional branch 180, a successive therapy subsession is commenced.

Eventually, in conducting the inquiry in decision diamond 178 it will be revealed to microprocessor 138 that therapy period T_{M} has fully transpired, or in other words that time T=T_{M}. As specified in procedure rectangle 182, the driver of light-emitting diode 67 in microprocessor 138 then operates light-emitting diode 67 in operative mode P(T=T_{M}) in order to alert the user that operation of medicament patch 16 is about to cease. Finally, as called for in procedure rectangle 184, the shutoff switch in microprocessor 138 turns voltage V off by disabling power source 62, and the illustrated methodology concludes in termination oval 188.

Performance measurement anomalies have been observed in actual waveforms of current output during the sampling operations of electronic circuitry 60.

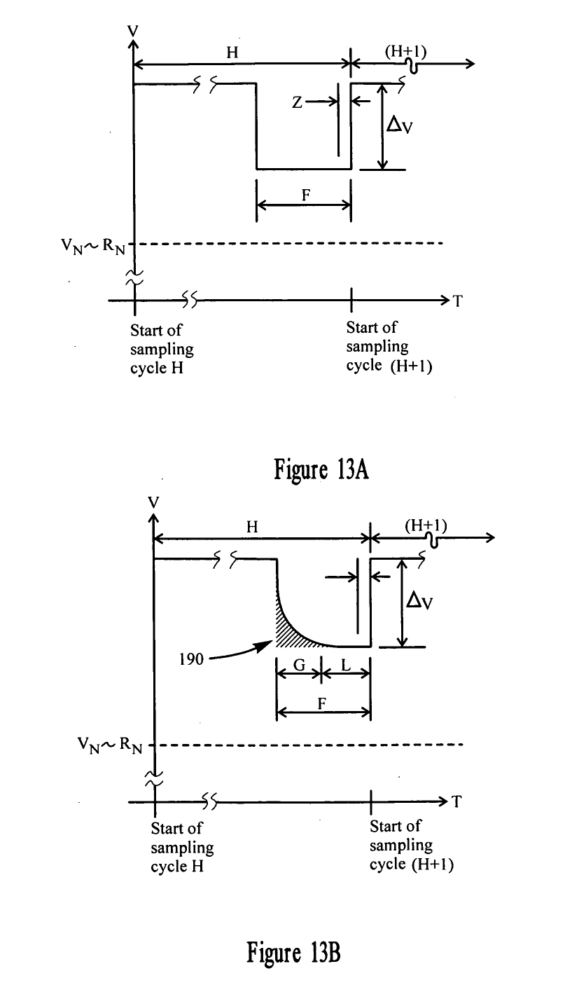

These anomalies are discovered by a comparison of the performance curves presented in FIGS. 13A and 13B. The measurement by electronic circuitry 60 of skin resistance R_{S} actually occurs as a measure of the voltage V applied to the skin by electronic circuitry 60. FIGS. 13A and 13B are performance curve of the voltage V applied to the skin by electronic circuitry 60, which is in turn interpreted as reflecting skin resistance R_{S}. Depicted in each is such a performance curve taken over a single typical sampling cycle H at the initiation of the operation of electronic circuitry 60.

At some point in time during the duration of sampling cycle H, the monitor of electronic circuitry 60 switches for a predetermined period of time, which is usually much shorter than the duration of sampling cycle H, from a pass-through mode of operation in which current from power source 62 is directed through the monitor to the skin of the patient, into a current-sampling mode of operation in which microprocessor 122 simultaneously determines the existing skin resistance R_{S} by measuring the amount of the voltage being applied to the skin.

What is expected during each sampling period H is an immediate drop of in the voltage applied to the skin as the internal workings of the monitor shift from the internal contacts required for the ongoing pass-through mode of operation into the temporary contacts effected in addition thereto for the sampling mode of operation. This would appear as a negative voltage square wave superimposed on the level voltage otherwise being applied during the pass-through mode of operation.

Instead, what is observed is illustratively depicted in FIG. 13A. The onset of the expected negative voltage square wave is postponed for a delay period G the voltage level detected by electronic circuitry 60 declines through a voltage drop ΔV and thereafter assumes a relatively constant voltage level during which accurate voltage sampling is feasible. The amount of voltage drop ΔV is interpreted as reflecting the degree of the reduction in skin resistance R_{S} from threshold skin resistance R_{∞} during the progress of electroporation. At some predetermined voltage drop ΔV, electronic circuitry 60 is programmed to consider that detected skin resistance R_{S} has declined to or below predetermined percentage P of threshold skin resistance R_{∞} to the value R_{N}. Then only can or does the administration of medicament begin.

Thus what is intended as possibly a generously ample amount of time in which to conduct sampling turns out to only be an apparent sampling duration F that is made up of an initial delay period G followed by an available accurate sampling window L. Available accurate sampling window L is the remainder of the time originally intended for sampling, and available accurate sampling window L is the only period during which actuate voltage sampling is possible. That voltage sampling is then conducted in an actual sampling period X, which is usually undertaken just prior to the conclusion of available accurate sampling window L.

The delay period G in arising in each sampling cycle H is highlighted as shaded region 190 in FIG. 13B. If the apparent sampling duration F is too short to allow the entire transition through delay period G to be completed, available accurate sampling window L ever exists. Accurate voltage sampling never occurs, and the possibility exists electronic circuitry 60 will never be able to turn on and deliver medicament.

From this behavior of electronic circuitry 60, it has been concluded that the engagement of the electrical contacts of an active medicament patch with the skin of a patient and the commencement of the application of a voltage differential between those contacts not only induces current flow through the skin, but also develops an appreciable amount of capacitance between the skin and each of those electrical contacts.

Apparently, not all electrical current leaving the electronic circuitry 60 actually becomes skin current I_{S} that is capable of transferring medicament. Some of the current leaving electronic circuitry 60 becomes a stored layer of charge on the electrical contact. This layer of charge in turn induces a layer of oppositely directed charge to accumulate in the skin surface.

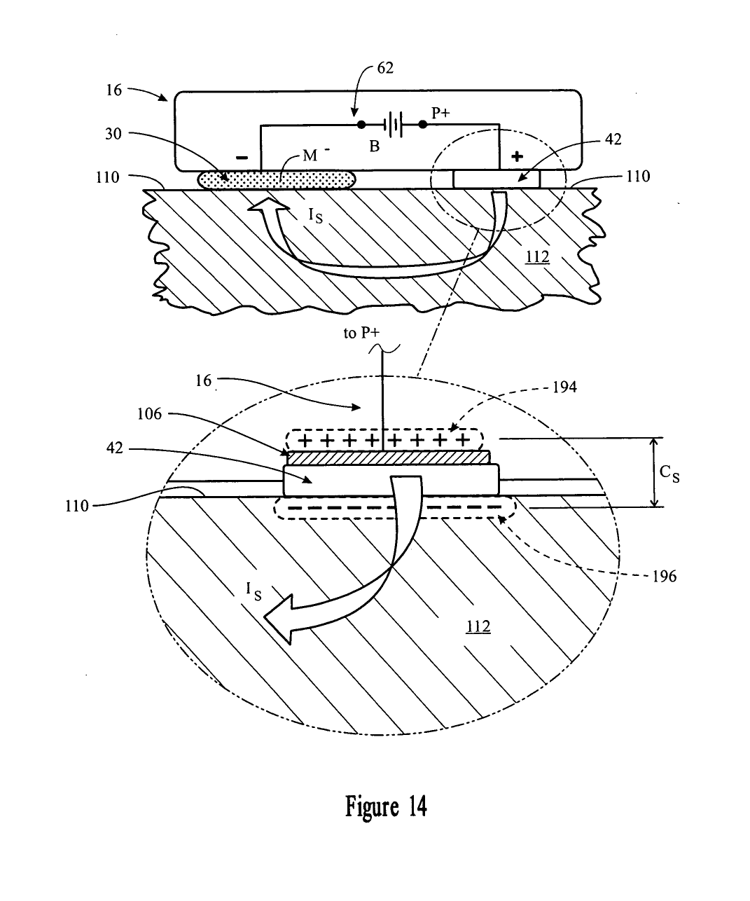

The situation is depicted diagrammatically in FIG. 14. There, a positive layer 194 of charge has collected on return electrode contact pad 106, and a responsive negative layer 196 has accumulated in skin 112 opposite from positive layer 194. The presence of positive layer 194 and negative layer 196 in such proximity gives rise in effect to a storage capacitance C_{S} therebetween. The size of storage capacitance C_{S} does not vary over time during steady state operation, but whenever electronic circuitry 60 implements the switching activity that permits electronic circuitry 60 to measure voltage, the stored charge that produces storage capacitance C_{S} disburses back into electronic circuitry 60, causing the delay in actual sampling detected as the performance measurement anomaly illustratively presented in FIG. 13B.

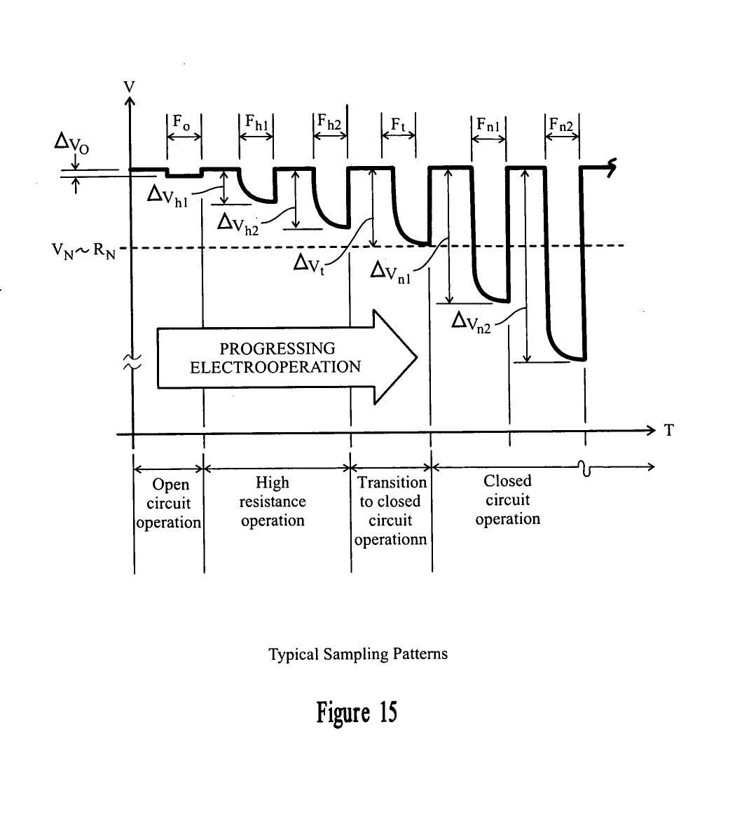

FIG. 15 is an illustrative performance curve in terms of voltage V corresponding to skin resistance R_{S} over a succession of typical sampling cycles of the type shown in FIG. 13B during the progression of electroporation at the initiation of the operation of electronic circuitry 60. Eventually, voltage drop ΔV comes to equal and exceed a voltage value that corresponds to a predetermined value of normal skin resistance R_{N} considered to correspond to a closed circuit through the skin. As skin resistance R_{S} decreases over time, the amount of each voltage drop ΔV increases.

The invention may be embodied in other specific forms without departing from the spirit or essential characteristics thereof. The described embodiments are to be considered in all respects only as illustrative and not restrictive. The scope of the invention is, therefore, to be defined by the appended claims, rather than by the foregoing description. All variations from the literal recitations of the claims that are, nonetheless, within the range of equivalency correctly attributable to the literal recitations are, however, to be considered to be within the scope of those claims.

What is claimed is:

1. A transdermal medicament patch comprising:
(a) a flexible substrate having a therapeutic face configured for releasable retention against the skin of a patient;
(b) a medicament matrix susceptible to permeation by medicament and secured to said therapeutic face of said substrate;
(c) a return electrode on secured to said therapeutic face spaced from said medicament matrix, said return electrode and said medicament matrix effecting electrically conductive engagement with the skin of the patient when said substrate is retained thereupon;
(d) a power source carried on said substrate and being so electrically coupled between said medicament matrix and said return electrode as to cause iontophoretic migration of medicament from said medicament matrix into the skin of the patient; and
(e) dosage control means carried non-removably on said substrate for limiting to a predetermined medicament quantity the total medicament administered into the skin of the patient by said iontophoretic migration during a predetermined therapy period.

2. A medicament patch as recited in claim 1, wherein said dosage control means comprises a medicament migration monitor, said medicament migration monitor periodically measuring the rate of said iontophoretic migration and correspondingly producing an output signal indicative of the instantaneous status of said iontophoretic migration.

3. A medicament patch as recited in claim 2, further comprising a user switch carried on said substrate, said switch permitting a user to initiate operation of said power source.

4. A medicament patch as recited in claim 3, wherein said dosage control means further comprises:
(a) a clock communicating with said medicament migration monitor, and activated by said user switch
(b) a dosage timer in said medicament migration monitor producing as an output signal a running cumulative total of the amount of medicament delivered into the skin of the patient by said iontophoretic migration; and
(c) a shutoff switch activatable by said medicament migration monitor to disable

5. A medicament patch as recited in claim 4, wherein:
(a) said power source is so electrically coupled between said medicament matrix and said return electrode as to cause said iontophoretic migration to occur at a substantially constant rate; and
(b) said medicament migration monitor activates said circuit breaker only when said output signal of said dosage timer equals the ratio of said predetermined medicament quantity divided by said substantially constant rate of iontophoretic migration.

6. A medicament patch as recited in claim 2, wherein said medicament migration monitor comprises:
(a) a voltage sampler coupled to said return electrode and producing as an output signal reflecting the electrical current flow resistance through the skin of the patient between said medicament matrix and said return electrode; and
(b) a signal comparator evaluating said output signal of said voltage sampler and classifying said electrical current flow resistance through the skin of the patient among a predetermined typography of possible electrical current flow resistances having relevance to the status of said iontophoretic migration.

7. A medicament patch as recited in claim 6, wherein said predetermined typography of possible electrical current flow resistances comprises the following classes of electrical current flow resistance:
(a) an extremely elevated skin resistance reliably understandable as signifying the existence of an open circuit at the skin of the patient;
(b) a high skin resistance reliably understandable as signifying the progress of skin electroporation; and
(c) a normal skin resistance reliably understandable as signifying the existence of a closed circuit through the skin of the patient between said medicament matrix and said return electrode

8. A medicament patch as recited in claim 6, wherein said voltage sampler comprises a sensing resistor electrically coupled between said return electrode and said power source.

9. A medicament patch as recited in claim 1, wherein said dosage control means comprises dosing verification means carried non-removably on said substrate for confirming to a user that said iontophoretic migration is occurring.

10. A medicament patch as recited in claim 9, wherein:
(a) said predetermined therapy period comprises a plurality of temporally non-contiguous therapy subsessions; and
(b) said dosing verification means comprises:
(i) a user-perceivable indicator; and
(ii) a driver for said indicator, said driver operating said indicator in a distinct delivery confirmation mode during each of said therapy subsessions, respectively.

11. An electrical circuit for managing the operation of an active transdermal medicament patch of the type including a substrate carrying a medicament matrix and a return electrode that each effect electrically-conductive engagement with the skin of a patient, said electrical circuit comprising:
(a) a power source carried on said substrate and being so electrically coupled between said medicament matrix and said return electrode as to cause iontophoretic migration of medicament from said medicament matrix into the skin of the patient to occur at a substantially constant rate;
(b) a microprocessor carried on said substrate and being electrically interposed between said power source and the return electrode, said microprocessor, said microprocessor comprising:
(i) an input contact coupled electrically to said power source;
(ii) an output contact coupled electrically to the return electrode; and
(iii) a monitoring contact at which to periodically measure the instantaneous rate of said iontophoretic migration; and
(c) a sensing resistor series connected between said monitoring contact of said microprocessor and the return electrode

12. An electric circuit as recited in claim 11, wherein:
(a) said microprocessor further comprises an activity indication contact; and
(b) said electrical circuit further comprises an indicator circuit capable of confirming to a user that said iontophoretic migration is occurring

13. An electric circuit as recited in claim 12, wherein said indicator circuit comprises:
(a) a light-emitting diode electrically coupled to said activity indication contact of said microprocessor; and
(b) a bias resistor series connected with said light-emitting diode between said activity indication contact and said input contact of said microprocessor.

14. An electric circuit as recited in claim 11, wherein said microprocessor comprises a read-only memory storing values of a predetermined typography of electrical current flow resistances relevant to the status of said iontophoretic migration.

15. An electric circuit as recited in claim 14, wherein said typography of electrical current flow resistances comprises:
(a) an extremely elevated skin resistance reliably understandable as signifying the existence of an open circuit at the skin of the patient;
(b) a high skin resistance reliably understandable as signifying the progress of skin electroporation; and
(c) a normal skin resistance reliably understandable as signifying the existence of a closed circuit through the skin of the patient between said medicament matrix and said return electrode

16. An electric circuit as recited in claim 14, wherein said microprocessor further comprises:
(a) a voltage sampler coupled to the return electrode and producing as an output signal reflecting the electrical current flow resistance through the skin of the patient between the medicament matrix and the return electrode; and
(b) a signal comparator evaluating said output signal of said voltage sampler and classifying said electrical current flow resistance through the skin of the patient among said typography of electrical current flow resistances stored in said read-only memory.

17. An electric circuit as recited in claim 16, wherein said voltage sampler ascertains a value of the electrical current flow resistance by reference to the voltage presented to the skin of the patient by the return electrode during a predetermined actual sampling period within an available accurate sampling window following a delay period of sufficient duration to allow for the dissipation of switching-related transients associated with developed skin capacitance at the medicaments matrix and at the return electrode.

18. An electric circuit as recited in claim 17, wherein said actual sampling period occurs immediately prior to the conclusion of said available accurate sampling window.

19. An electric circuit as recited in claim 17, wherein said delay period is temporally contiguous with said available accurate sampling window.

20. An electric circuit as recited in claim 14, further comprising a user switch carried on the substrate, operation of said user switch initiating operation of said power source.

21. An electric circuit as recited in claim 18, further comprising
(a) a clock activated by said user switch;
(b) a dosage timer in said microprocessor producing as an output signal a running cumulative total of the amount of medicament delivered to the skin of the patient by said iontophoretic migration; and
(c) a shutoff switch activatable by said microprocessor to disable said power source.

22. A transdermal medicament patch comprising:
(a) a flexible substrate having a therapeutic face configured for releasable retention against the skin of a patient;
(b) a medicament matrix susceptible to permeation by medicament and secured to said therapeutic face of said substrate;
(c) a return electrode on secured to said therapeutic face spaced from said medicament matrix, said return electrode and said medicament matrix effecting electrically conductive engagement with the skin of the patient when said substrate is retained thereupon;
(d) a power source carried on said substrate and being electrically coupled between said medicament matrix and said return electrode; and
(d) therapy status advisement means non-removably carried on said substrate and driven by said power source for communicating to a user the extent of completion of a predetermined therapy period wherein medicament is to be administered from said medicament matrix into the skin of the patient using iontophoretic migration.

23. A medicament patch as recited in claim 22, wherein said therapy status advisement means comprises a visual indicator.

24. A medicament patch as recited in claim 23, wherein said therapy status advisement means comprises:
(a) a light-emitting diode; and
(b) a driver for said light-emitting diode, said driver causing said light-emitting diode to operate in various of a plurality of preselected modes from the activation of said power source until the completion of the predetermined therapy period.

25. A medicament patch as recited in claim 23, wherein said therapy status advisement means comprises:
(a) a light-emitting diode;
(b) a timer active during said therapy period; and
(c) a driver for said light-emitting diode, said driver causing said light-emitting diode to operate in various of a plurality of preselected modes when said timer is active.

26. A medicament patch as recited in claim 25, wherein said driver causes said light-emitting diode to operate intermittently.

27. A medicament patch as recited in claim 25, wherein said timer is deactivated when said iontophoretic migration is absent.

28. A medicament patch as recited in claim 27, wherein said driver causes said light-emitting diode to operate in an open circuit mode when said timer is deactivated prior to the end of said therapy period.

29. A medicament patch as recited in claim 25, wherein said therapy period comprises a sequence of non-overlapping predetermined therapy subsessions, and said driver causes said light-emitting diode to operate in a distinct delivery confirmation mode during each of said therapy subsessions, respectively.

30. A medicament patch as recited in claim 29, wherein said driver causes said light-emitting diode to operate in a transition mode at the end of selected of said therapy subsessions.

31. A medicament patch as recited in claim 29, wherein said selected of said therapy subsessions comprises the final of said therapy stages.
